(19) United States
(12) Patent Application Publication
(10) Pub. No.: US 2023/0174649 A1
(43) Pub. Date: Jun. 8, 2023

(54) PHARMACEUTICAL COMPOSITIONS CONTAINING ANTI-CD47 ANTIBODIES

(71) Applicant: I-MAB BIOPHARMA US LIMITED, Gaithersburg, MD (US)

(72) Inventors: Wei CAO, Shanghai (CN); Chuanyan YE, Shanghai (CN); Zhengyi WANG, Shanghai (CN); Bingshi GUO, Shanghai (CN)

(21) Appl. No.: 17/997,522

(22) PCT Filed: Apr. 29, 2021
(86) PCT No.: PCT/CN2021/091103

§ 371 Date: Oct. 28, 2022

(30) Foreign Application Priority Data
Apr. 30, 2020 (CN) PCT/CN2020/088226

(51) Int. Cl. C07K 16/28; A61P 35/00

(52) U.S. Cl. CPC C07K 16/2803; C07K 16/2887; A61P 35/00; A61K 2039/507

(57) ABSTRACT

Provided is a pharmaceutical composition comprising an isolated anti-CD47 antibody or an immunologically active fragment thereof, a therapeutic agent and a pharmaceutically acceptable carrier. These pharmaceutical compositions are synergistic by enhancing phagocytosis of cancer cells as compared to the use of single agents. In addition, also provided is a method for treating a disease in a human subject in need thereof by administering the pharmaceutical composition.

### CROSS-REFERENCE TO RELATED APPLICATION

This application claims priority to international application number PCT/CN2020/088226, filed on Apr. 30, 2020, the contents of which are incorporated herein by reference in their entirety.

### BACKGROUND OF THE INVENTION

CD47 (Cluster of Differentiation 47) was first identified as a tumor antigen on human ovarian cancer in the 1980s. Since then, CD47 has been found to be expressed on multiple human tumor types including acute myeloid leukemia (AML), chronic myeloid leukemia, acute lymphoblastic leukemia (ALL), non-Hodgkin's lymphoma (NHL), multiple myeloma (MM), bladder cancer, and other solid tumors. High levels of CD47 allow cancer cells to avoid phagocytosis despite having a higher level of calreticulin—the dominant pro-phagocytic signal.

Also known as integrin-associated protein (IAP), ovarian cancer antigen OA3, Rh-related antigen and MERG, CD47 is a multi-spanning transmembrane protein belonging to the immunoglobulin (Ig) superfamily that is universally expressed on mammalian cells and tissues. Through its interactions with signal-regulatory proteins alpha (SIRPα), an inhibitory protein expressed on macrophages, CD47 triggers tyrosine phosphorylation in the SIRPα cytoplasmic immunoreceptor tyrosine-based inhibitory motifs (ITIMs) and recruitment of protein tyrosine phosphatases SHP-1/SHP-2, which further mediates negative signaling events that inhibit macrophage phagocytosis. For this, CD47 acts as a “self” marker and a “do not eat me” signal, preventing macrophage engulfment of host cells. The interactions between CD47 and SIRPα play a critical role in restraining macrophages.

For this reason, blood cells, such as red blood cells, platelets, and lymphocytes, express CD47 on their surface to protect themselves from rapid elimination by splenic macrophages. However, CD47 is expressed at even higher levels on leukemia stem cells (LSCs) than their normal counterparts. Higher expression levels of CD47 on human LSCs contribute to pathogenesis by inhibiting their phagocytosis through the interaction of CD47 with SIRPα. Accumulating evidence suggests that CD47 expression on human solid tumor cells is a common mechanism through which these cancer cells protect themselves from phagocytosis, allowing tumor cell proliferation and metastasis (See: Frontiers in immunology, April 2017, Volume 8, Article 404).

Therefore, interruption of the CD47-SIRPα pathway by anti-CD47 antibodies might have a therapeutic effect to enhance cancer cell phagocytic uptake. As disclosed in the International Application No. PCT/US2017/057535, we have provided novel anti-CD47 antibodies or an immunologically active fragments thereof that binds human CD47, thereby preventing human CD47 from interacting with SIRPα and promoting macrophage-mediated phagocytosis of a CD47-expressing cell. Very significantly, it does not cause a significant level of hemagglutination or depletion of red blood cells.

By contrast to the antiphagocytic (don't-eat-me) signal CD47 on tumor cells, accumulated evidences indicate that cell surface calreticulin is considered as an “eat-me” signal and facilitates phagocytic uptake of cancer cells by immune system. Clarke and Smyth demonstrated that drug treatments (anthracyclines) caused tumor cell to expose a surface prophagocytic protein, calreticulin, which induced immunogenic cell death (See: Nature Biotechnology. 2007; 25(2):192-193). Therefore, calreticulin-mediated immune mechanisms might be an important strategy for developing new anticancer therapy.

Therapeutic monoclonal antibodies have proven clinically important in the treatment of cancer, however, there still remains considerable needs in manipulating and promoting phagocytosis of tumor cell. The present invention satisfies these, and other, needs.

### BRIEF SUMMARY OF THE INVENTION

Methods and pharmaceutical compositions are provided for disease treatment in this invention. In this pharmaceutical composition, one essential component is a novel CD47 antibody (disclosed in PCT/US2017/057535) that blocks CD47 on the cell surface and prevents interactions between CD47 and SIRPα. Another essential component is a second therapeutic agent that can be a small molecule therapeutic agent for increasing the expression level of calreticulin and/or inhibiting the expression levels of CD47, or a large molecule (e.g., a second antibody such as CD20 antibody) with synergistic effect. In some embodiments, the pharmaceutical composition comprises three essential components, i.e., a novel CD47 antibody, a small molecule therapeutic agent and a second antibody with synergistic effect (such as CD20 antibody). The pharmaceutical compositions are synergistic in promoting phagocytosis of cancer cells as compared to the use of any single agent. The combination of agents is particularly useful in the treatment of cancer, a fibrotic disease, a disease related to inhibition of phagocytosis, or a disease related to platelet aggregation.

In some embodiments, this invention provides a pharmaceutical composition, comprising a novel CD47 antibody or an immunologically active fragment, a therapeutic agent and a pharmaceutically acceptable carrier or excipient, in which the isolated antibody or an immunologically active fragment comprises a heavy chain variable region (VH) having amino acid sequence that is at least 95% identical to an amino acid sequence selected from the group consisting of SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, SEQ ID NO: 67, SEQ ID NO: 69, SEQ ID NO: 71, SEQ ID NO: 73, SEQ ID NO: 75, SEQ ID NO: 77, SEQ ID NO: 79, and SEQ ID NO: 81; and a light chain variable region (VL) having amino acid sequence that is at least 95% identical to an amino acid sequence selected from the group consisting of SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 70, SEQ ID NO: 72, SEQ ID NO: 74, SEQ ID NO: 76, SEQ ID NO: 78, SEQ ID NO: 80, and SEQ ID NO: 82.

In some another embodiments, the isolated antibody or an immunologically active fragment thereof comprises a VH/VL pair, wherein the VH/VL pair comprises VH and VL chain sequences that are respectively at least 95% identical to amino acid sequences selected from the group consisting of SEQ ID NO: 1 and SEQ ID NO: 2 (i.e., 1A1), SEQ ID NO: 3 and SEQ ID NO: 4 (i.e., 1F8), SEQ ID NO: 5 and SEQ ID NO: 6 (i.e., 2A11), SEQ ID NO: 7 and SEQ ID NO: 8 (i.e., 2C2), SEQ ID NO: 9 and SEQ ID NO:10 (i.e., 2D7), SEQ ID NO: 11 and SEQ ID NO: 12 (i.e., 2G4), SEQ ID NO: 13 and SEQ ID NO: 14 (i.e., 2G11), SEQ ID NO: 15 and SEQ ID NO: 16 (i.e., 6F4), SEQ ID NO: 17 and SEQ ID NO: 18 (i.e., 5H1), SEQ ID NO: 19 and SEQ ID NO: 20 (i.e., 5F6), SEQ ID NO: 21 and SEQ ID NO: 22 (i.e., 1F3), SEQ ID NO: 23 and SEQ ID NO: 24 (i.e., 2A4), SEQ ID NO: 25 and SEQ ID NO: 26 (i.e., 2B12), SEQ ID NO: 27 and SEQ ID NO: 28 (i.e., 13A11), SEQ ID NO: 29 and SEQ ID NO: 30 (i.e., 15E1), SEQ ID NO: 31 and SEQ ID NO: 32 (i.e., 13H3), SEQ ID NO: 33 and SEQ ID NO: 34 (i.e., 14A8), SEQ ID NO: 35 and SEQ ID NO: 36 (i.e., 16H3), SEQ ID NO: 37 and SEQ ID NO: 38 (i.e., 1A1), SEQ ID NO: 39 and SEQ ID NO: 40 (i.e., 1A1-A), SEQ ID NO: 41 and SEQ ID NO: 42 (i.e., 1A1-Q), SEQ ID NO: 43 and SEQ ID NO: 44 (i.e., 1A2), SEQ ID NO: 45 and SEQ ID NO: 46 (i.e., 1A8), SEQ ID NO: 47 and SEQ ID NO: 48 (i.e., 1B1), SEQ ID NO: 49 and SEQ ID NO: 50 (i.e., 1B2), SEQ ID NO: 51 and SEQ ID NO: 52 (i.e., 1H3), SEQ ID NO: 53 and SEQ ID NO: 54 (i.e., 1H3-Q), SEQ ID NO: 55 and SEQ ID NO: 56 (i.e., 1H3-A), SEQ ID NO: 57 and SEQ ID NO: 58 (i.e., 2A2), SEQ ID NO: 59 and SEQ ID NO: 60 (i.e., 2A3), SEQ ID NO: 61 and SEQ ID NO: 62 (i.e., 2A6), SEQ ID NO: 63 and SEQ ID NO: 64 (i.e., 2A10), SEQ ID NO: 65 and SEQ ID NO: 66 (i.e., 2B1), SEQ ID NO: 67 and SEQ ID NO: 68 (i.e., 2C6), SEQ ID NO: 69 and SEQ ID NO: 70 (i.e., 2E7), SEQ ID NO: 71 and SEQ ID NO: 72 (i.e., 2E9), SEQ ID NO: 73 and SEQ ID NO: 74 (i.e., 2F1), SEQ ID NO: 75 and SEQ ID NO: 76 (i.e., 2F3), SEQ ID NO: 77 and SEQ ID NO: 78 (i.e., 34C5), SEQ ID NO: 79 and SEQ ID NO: 80 (i.e., A1A), and SEQ ID NO: 81 and SEQ ID NO: 82 (i.e., T4J).

In some other embodiments, the isolated antibody or an immunologically active fragment that specifically binds human CD47 comprises a variable heavy (VH) chain sequence that is at least 95% identical to an amino acid sequence selected from the group consisting of SEQ ID NO: 3, SEQ ID NO: 31, SEQ ID NO. 79, and SEQ ID NO: 81; and a variable light (VL) chain sequence that is at least 95% identical to an amino acid sequence selected from the group consisting of SEQ ID NO: 4, SEQ ID NO: 32, SEQ ID NO: 80, and SEQ ID NO: 82.

In another aspect, the present invention provides a pharmaceutical composition, comprising a novel CD47 antibody or an immunologically active fragment, a therapeutic agent and a pharmaceutically acceptable carrier or excipient, in which the isolated antibody or an immunologically active fragment comprises a VH chain having VH CDR1, VH CDR2 and VH CDR3 of the sequences shown below, and a VL chain having VL CDR1, VL CDR2, and VL CDR3 of the sequences shown below:
VH CDR1: NAWMN (SEQ ID NO: 85) or RAWMN (SEQ ID NO: 86)
VH CDR2: RIKRKTDGETTDYAAPVKG (SEQ ID NO: 87)
VHCDR3: SNRAFDI (SEQ ID NO: 88)
VL CDR1: KSSQSVLYAGNNRNYLA (SEQ ID NO: 89) or KSSQSVLYAGNNRNYLA (SEQ ID NO: 90)
VL CDR2: QASTRAS (SEQ ID NO: 91)
VL CDR3: QQYYTPPLA (SEQ ID NO: 92)

In some other embodiments, the isolated antibody or an immunologically active fragment that specifically binds human CD47 comprises a VH/VL pair, in which the VH/VL pair comprises VH and VL chain sequences that are respectively at least 95% identical to amino acid sequences selected from the group consisting of SEQ ID NO: 3 and SEQ ID NO: 4 (i.e., 1F8), SEQ ID NO: 31 and SEQ ID NO: 32 (i.e., 13H3), SEQ ID NO: 79 and SEQ ID NO: 80 (i.e., A1A); and SEQ ID NO: 81 and SEQ ID NO: 82 (i.e., T4J). Antibodies 13H3 and A1A are affinity matured clones of antibody 1F8. Amino acid sequences of the three antibodies are highly similar.

In further embodiments, the isolated antibody or an immunologically active fragment thereof that specifically binds human CD47 includes a heavy chain of SEQ ID NO: 81 and a light chain of SEQ ID NO: 82.

The isolated antibody or an immunologically active fragment can be chimeric or humanized. And the isolated antibody can be a monoclonal antibody, a bispecific antibody or a fusion antibody that binds human CD47. They can prevent or significantly reduce human CD47 from interacting with SIRPα, or promotes macrophage-mediated phagocytosis of a CD47-expressing cell. The CD47 antibodies of this invention do not cause a significant or noticeable level of hemagglutination or depletion of red blood cells, and in many cases, they do not cause hemagglutination or depletion of red blood cells at all.

In one aspect, the isolated bispecific antibody comprises a first arm and a second arm. The first arm comprises the antibody or an immunologically active fragment thereof that binds human CD47, and the second arm comprises a second monoclonal antibody that does not bind human CD47.

In another aspect, the second arm of the isolated bispecific antibody binds to cancer cell.

In yet another aspect, the fusion antibody is the isolated antibody or an immunologically active fragment thereof conjugated with an additional protein, a small-molecule agent or a marker.

In yet another aspect, the additional protein is an antibody or a cytokine. The small molecule agent is an anti-cancer or anti-inflammation agent. And the marker is a biomarker or fluorescent marker.

In some embodiments, the therapeutic agent in the pharmaceutical composition is a small molecule chemotherapeutic agent, which does not cause substantial toxicity to macrophages. As used herein, the term “substantial toxicity” means toxicity of considerable concern because of (a) the seriousness of the toxicity effect, and (b) the fact or probability of its occurrence. These therapeutic agents in the pharmaceutical composition can synergize with the anti-CD47 antibodies to increase the macrophage phagocytosis effect by increasing the expression level of calreticulin and/or decrease the expression level of CD47, or provide additive effect to anti-CD47 antibody.

In one aspect, the therapeutic agent increases expression level of Calreticulin. In another aspect, the therapeutic agent inhibits expression level of CD47.

In some embodiments, the therapeutic agent in the pharmaceutical composition is a chemotherapeutic agent. Such a chemotherapeutic agent can be a small molecule drug and can be Azacitidine, Venetoclax or Copanilisib. In some other embodiments, the therapeutic agent is Azacitidine or Venetoclax. In some other embodiments, the therapeutic agent is Azacitidine.

In yet another embodiment, the therapeutic agent in the pharmaceutical composition is a second antibody or an immunologically active fragment thereof. In some further embodiments, this second antibody selectively binds CD20 (thereby called “CD20 antibody” or “anti-CD20 antibody”) and can promote phagocytic elimination of cancer cell. In a further embodiment, the CD47 antibody or an immunologically active fragment synergize the CD20 antibody promoting phagocytic elimination of cancer cell.

In yet another embodiment, the additional/second antibody that selectively binds CD20 is Rituximab (of SEQ ID NO: 83 and SEQ ID NO: 84) or its biosimilar, and the second antibody, such as Rituximab (of SEQ ID NO: 83 and SEQ ID NO: 84), can synergize with a CD47 antibody to facilitate the phagocytosis of tumor cell.

In yet another embodiment, the therapeutic agent in the pharmaceutical composition comprises both the small molecular chemotherapeutic agent and the second antibody that binds CD20. In some further embodiments, the therapeutic agent comprises both Azacitidine and Rituximab as synergistic agents. In another further embodiments, the therapeutic agent comprises Venetoclax and Rituximab as synergistic agents.

The present invention also provides a method for treating diseases in a subject using the pharmaceutical composition. Examples of diseases include, but are not limited to, cancer, a fibrotic disease, a disease related to inhibition of phagocytosis, or a disease related to platelet aggregation.

Examples of cancer are, but not limited to, ovarian cancer, colon cancer, breast cancer, lung cancer, head and neck cancer, bladder cancer, colorectal cancer, pancreatic cancer, non-Hodgkin's lymphoma, acute lymphocytic leukemia, chronic lymphocytic leukemia, acute myeloid leukemia, chronic myelogenous leukemia, hairy cell leukemia (HCL), T-cell prolymphocytic leukemia (T-PLL), large granular lymphocytic leukemia, adult T-cell leukemia, multiple myeloma, melanoma, leiomyoma, leiomyosarcoma, glioma, glioblastoma, myelomas, monocytic leukemias, B-cell derived leukemias, T-cell derived leukemias, B-cell derived lymphomas, T-cell derived lymphomas, endometrial cancer, kidney cancer, melanoma, prostate cancer, thyroid cancer, cervical cancer, gastric cancer, liver cancer, and solid tumors; the fibrotic disease is selected from the group consisting of: myocardial infarction, angina, osteoarthritis, pulmonary fibrosis, asthma, cystic fibrosis, bronchitis, and asthma; the disease related to inhibition of phagocytosis is a cardiovascular disease; the disease related to platelet aggregation is Glanzmann Thrombasthenia, prolonged bleeding time, immune thrombocytopenia (ITP), von Willebrand disease (vWD).

Examples of cardiovascular disease are, but not limited to, atherosclerosis, stroke, hypertensive heart disease, rheumatic heart disease, cardiomyopathy, heart arrhythmia, congenital heart disease, valvular heart disease, carditis, aortic aneurysms, peripheral artery disease, and venous thrombosis.

The present invention further provides uses of the pharmaceutical composition for the manufacture of a medicament for treatment of diseases.

### BRIEF DESCRIPTIONS OF THE DRAWINGS

FIG. 1 shows the viability of macrophages after being treated with selected small molecule therapeutic agents for 16 hours.

FIG. 2 shows the expression levels of CD47 and Calreticulin. In this assay, Toledo cells were treated with selected small molecule therapeutic agents for 12 hours and then the CD47 (left) and Calreticulin (right) expression level were assessed by FACS assay.

FIG. 3 shows the phagocytic ability of macrophages after being co-cultured with tumor cells in the presence of the isolated anti-CD47 antibody, Rituximab and selected small molecule therapeutic agents for 2-6 hours. The phagocytosis was analyzed by FACS assay.

FIG. 4 shows tumor growth over time in four different treatment groups.

FIG. 5 shows changes of tumor body weight in four different treatment groups.

FIG. 6 shows tumor body weight changes in different treatment groups using female NOG mice bearing subcutaneous HL-60 xenograft model. FIG. 6a shows tumor body weight changes and FIG. 6b shows the change in percentage (%).

FIG. 7 shows tumor growth curves in different treatment groups using female NOG mice bearing subcutaneous HL-60 xenograft model.

### DETAILED DESCRIPTION OF THE INVENTION

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Unless defined otherwise, all technical and scientific terms used herein have the same meaning as commonly understood by one of ordinary skill in the art to which this invention belongs.

As used herein, the term “antibody” is used in the broadest sense and specifically covers monoclonal antibodies (including full length monoclonal antibodies), polyclonal antibodies, multi-specific antibodies (e.g., bispecific antibodies), and antibody fragments so long as they exhibit the desired biological activity. “Antibodies'” (or “Abs”) and “immunoglobulins” (or “Igs”) are glycoproteins having the same structural characteristics. While antibodies exhibit binding specificity to a specific antigen, immunoglobulins include both antibodies and other antibody-like molecules which lack antigen specificity.

As used herein, the term “immunologically active fragment”, and all grammatical variants thereof, are defined as a portion of an intact antibody comprising the antigen binding site or variable region of the intact antibody, wherein the portion is free of the constant heavy chain domains (i.e. CH2, CH3, and CH4, depending on antibody isotype) of the Fc region of the intact antibody. Examples of antibody fragments include Fab, Fab′, Fab′-SH, F(ab′)2, and Fv fragments; diabodies; any antibody fragment that is a polypeptide having a primary structure consisting of one uninterrupted sequence of contiguous amino acid residues (referred to herein as a “single-chain antibody fragment” or “single chain polypeptide”), including without limitation (1) single-chain Fv (scFv) molecules, (2) single chain polypeptides containing only one light chain variable domain, or a fragment thereof that contains the three CDRs of the light chain variable domain, without an associated heavy chain moiety, and (3) single chain polypeptides containing only one heavy chain variable region, or a fragment thereof containing the three CDRs of the heavy chain variable region, without an associated light chain moiety; and multi-specific or multivalent structures formed from antibody fragments. In an antibody fragment comprising one or more heavy chains, the heavy chain(s) can contain any constant domain sequence (e.g. CHI in the IgG isotype) found in a non-Fc region of an intact antibody, and/or can contain any hinge region sequence found in an intact antibody, and/or can contain a leucine zipper sequence fused to or situated in the hinge region sequence or the constant domain sequence of the heavy chain(s).

As used herein, the term “monoclonal antibody” (mAb) refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Each mAb is directed against a single determinant on the antigen. In addition to their specificity, the monoclonal antibodies are advantageous in that they can be synthesized by hybridoma culture, uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made in an immortalized B cell or hybridoma thereof, or may be made by recombinant DNA methods.

As used herein, the term “an immunologically active fragment” of an antibody refers to a fragment of an antibody that exhibits immunologically active effect similar to that of the entire antibody. It is also referred to as “an antigen-binding fragment” of an antibody.

The monoclonal antibodies herein include hybrid and recombinant antibodies produced by splicing a variable (including hypervariable) domain of an CD47 antibody with a constant domain (e.g. “humanized” antibodies), or a light chain with a heavy chain, or a chain from one species with a chain from another species, or fusions with heterologous proteins, regardless of species of origin or immunoglobulin class or subclass designation, as well as antibody fragments (e.g., Fab, F(ab′)2, and Fv), so long as they exhibit the desired biological activity.

The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity.

As used herein, an “isolated” antibody is one which has been identified and separated and/or recovered from a component of its natural environment. Contaminant components of its natural environment are materials which would interfere with diagnostic or therapeutic uses for the antibody, and may include enzymes, hormones, and other proteinaceous or nonproteinaceous solutes. In some embodiments, the antibody will be purified (1) to greater than 75% by weight of antibody as determined by the Lowry method, and most preferably more than 80%, 90% or 99% by weight, or (2) to homogeneity by SDS-PAGE under reducing or nonreducing conditions using Coomassie blue or, preferably, silver stain. Isolated antibody includes the antibody in situ within recombinant cells since at least one component of the antibody's natural environment will not be present. Ordinarily, however, isolated antibody will be prepared by at least one purification step.

As used herein, the term “treatment” or “treating” refers to both therapeutic treatment and prophylactic or preventative measures of a disease (such as cancer or a fibrotic disease). Those in need of treatment include those already with the disease as well as those in which the disease is to be prevented.

Examples of cancer include, but are not limited to, ovarian cancer, colon cancer, breast cancer, lung cancer, head and neck cancer, bladder cancer, colorectal cancer, pancreatic cancer, non-Hodgkin's lymphoma, acute lymphocytic leukemia, chronic lymphocytic leukemia, acute myeloid leukemia, chronic myelogenous leukemia, hairy cell leukemia (HCL), T-cell prolymphocytic leukemia (T-PLL), large granular lymphocytic leukemia, adult T-cell leukemia, multiple myeloma, melanoma, leiomyoma, leiomyosarcoma, glioma, glioblastoma, myelomas, monocytic leukemias, B-cell derived leukemias, T-cell derived leukemias, B-cell derived lymphomas, T-cell derived lymphomas, endometrial cancer, kidney cancer, melanoma, prostate cancer, thyroid cancer, cervical cancer, gastric cancer, liver cancer, and solid tumors. The fibrotic disease can be myocardial infarction, angina, osteoarthritis, pulmonary fibrosis, asthma, cystic fibrosis, bronchitis, and asthma. The disease related to inhibition of phagocytosis can be a cardiovascular disease; and the disease related to platelet aggregation can be Glanzmann Thrombasthenia, prolonged bleeding time, immune thrombocytopenia (ITP), von Willebrand disease (vWD).

As used herein, the term “pharmaceutically acceptable carrier or excipient” refers to a carrier or an excipient that is useful for preparing a pharmaceutical composition or formulation that is generally safe, non-toxic, and neither biologically nor otherwise undesirable. A carrier or excipient employed is typically one suitable for administration to human subjects or other mammals. In making the compositions, the active ingredient is usually mixed with, diluted by, or enclosed with a carrier or excipient. When the carrier or excipient serves as a diluent, it can be a solid, semi-solid, or liquid material, which acts as a vehicle, carrier, or medium for the active ingredient of the antibody.

The CD47 antibodies of the invention can be bound to many different carriers and used to detect the presence of CD47 expressing cells. Examples of well-known carriers include glass, polystyrene, polypropylene, polyethylene, dextran, nylon, amylases, natural and modified celluloses, polyacrylamides, agaroses and magnetite. The nature of the carrier can be either soluble or insoluble for purposes of the invention. Those skilled in the art will know of other suitable carriers for binding monoclonal antibodies, or will be able to ascertain such, using routine experimentation.

The terms “pharmaceutically acceptable”, “physiologically tolerable” and grammatical variations thereof, as they refer to compositions, carriers, diluents and reagents, are used interchangeably and represent that the materials are capable of administration to or upon a human without the production of undesirable physiological effects to a degree that would prohibit administration of the composition.

Unless specifically indicated to the contrary, the term “conjugate” and “conjugated” used herein is defined as a heterogeneous molecule formed by the covalent attachment of one or more antibody fragment(s) to one or more polymer molecule(s), wherein the heterogeneous molecule is water soluble, i.e. soluble in physiological fluids such as blood, and wherein the heterogeneous molecule is free of any structured aggregate.

The term “chemotherapeutic agent” is a broad one covering many chemotherapeutic agents having different mechanisms of action. The chemotherapeutic agents that can be administered in combination with an anti-CD47 agent include, without limitation, azacitidine, idelalisib, duvelisib, venetoclax, copanlisib, lbrutinib, bendamustine, and lenalidomide.

The additional monoclonal antibodies that can be included in the present pharmaceutical composition is an antibody selectively binds CD20, which may include, without limitation, Rituximab which has a heavy chain of the following sequence:

(SEQ ID NO: 83)
QVQLQQPGAELVKPGASVKMSCKASGYTFTSYNMHWVKQTPGRGLEWIG
AIYPGNGDTSYNQKFKGKATLTADKSSSTAYMQLSSLTSEDSAVYYCAR
STYYGGDWYFNVWGAGTTVTVSAASTKGPSVFPLAPSSKSTSGGTAALG
CLVKDYFPEPVTVSWNSGALTSGVHTFPAVLQSSGLYSLSSVVTVPSSS
LGTQTYICNVNHKPSNTKVDKKAEPKSCDKTHTCPPCPAPELLGGPSVF
LFPPKPKDTLMISRTPEVTCVVVDVSHEDPEVKFNWYVDGVEVHNAKTK
PREEQYNSTYRVVSVLTVLHQDWLNGKEYKCKVSNKALPAPIEKTISKA
KGQPREPQVYTLPPSRDELTKNQVSLTCLVKGFYPSDIAVEWESNGQPE
NNYKTTPPVLDSDGSFFLYSKLTVDKSRWQQGNVFSCSVMHEALHNHYT
QKSLSLSPGK

and a light chain of the following sequence:

(SEQ ID NO: 84)
QIVLSQSPAILSASPGEKVTMTCRASSSVSYIHWFQQKPGSSPKPWIYA
TSNLASGVPVRFSGSGSGTSYSLTISRVEAEDAATYYCQQWTSNPPTFG
GGTKLEIKRTVAAPSVFIFPPSDEQLKSGTASVVCLLNNFYPREAKVQW
KVDNALQSGNSQESVTEQDSKDSTYSLSSTLTLSKADYEKHKVYACEVT
HQGLSSPVTKSFNRGEC

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

Although any methods and materials similar or equivalent to those described herein can also be used in the practice or testing of the present invention, the preferred methods are described in the Examples and the materials are now described below.

Materials used in the following examples included:
CellTrace™ Violet: Thermo Fisher Scientific, Catalog No.: C34557
CellTrace™ Far Red: Thermo Fisher Scientific, Catalog No.: 34564
CellTiter-Glo™ Luminescent Cell Viability Assay Kit: Promega, Catalog No.: G7573
Anti-calreticulin antibody: Abcam, Catalog No.: ab83220
Alexa Fluor® 647 Mouse Anti-Human CD47: BD biosciences, Catalog No.: 561249
Ficoll-Paque Plus: Axis-Shield, Catalog No.: AS1114547
MACS system: Miltenyi Biotech, Catalog No.: 130-045-201.
Tumor cell line, Toledo, purchased from ATCC.

As examples, a CD47 antibody suitable for the compositions of this invention would include (a) a variable heavy chain (VH) sequence that is selected from the group consisting of SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, SEQ ID NO: 67, SEQ ID NO: 69, SEQ ID NO: 71, SEQ ID NO: 73, SEQ ID NO: 75, SEQ ID NO: 77, SEQ ID NO: 79, and SEQ ID NO: 81; and (b) a variable light (VL) chain sequence that is selected from the group consisting of SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 70, SEQ ID NO: 72, SEQ ID NO: 74, SEQ ID NO: 76, SEQ ID NO: 78, SEQ ID NO: 80, and SEQ ID NO: 82.

In some instance, the isolated antibody or an immunologically active fragment that specifically binds human CD47 comprises a variable heavy (VH) chain sequence that is at least 95% identical to an amino acid sequence selected from the group consisting of SEQ ID NO: 3, SEQ ID NO: 31, SEQ ID NO. 79, and SEQ ID NO: 81; and a variable light (VL) chain sequence that is at least 95% identical to an amino acid sequence selected from the group consisting of SEQ ID NO: 4, SEQ ID NO: 32, SEQ ID NO: 80, and SEQ ID NO: 82; wherein the isolated monoclonal antibody or an immunologically active fragment thereof comprising a VH CDR1, VH CDR2 and VH CDR3 of the VH sequence shown in SEQ ID NO: 3, SEQ ID NO: 31, SEQ ID NO. 79, or SEQ ID NO: 81, and a VL CDR1, VL CDR2 and VL CDR3 of the VL sequence shown in SEQ ID NO: 4, SEQ ID NO: 32, SEQ ID NO: 80, or SEQ ID NO: 82; wherein the VH CDR1 having the amino acid sequence of NAWMN (SEQ ID NO: 85) or RAWMN (SEQ ID NO: 86), the VH CDR2 having the amino acid sequence of RIKRKTDGETTDYAAPVKG (SEQ ID NO: 87), and the VH CDR3 having the amino acid sequence of SNRAFDI (SEQ ID NO: 88); wherein the VL CDR1 having the amino acid sequence of KSSQSVLYSSNNRNYLA (SEQ ID NO: 89) or KSSQSVLYAGNNRNYLA (SEQ ID NO: 90), the VL CDR2 having the amino acid sequence of QASTRAS (SEQ ID NO: 91), and the VL CDR3 having the amino acid sequence of QQYYTPPLA (SEQ ID NO: 92).

In some further instance, a CD47 antibody suitable for the compositions of this invention would include a combined VH/VL chain sequence that is selected from the group consisting of SEQ ID NO: 1 and SEQ ID NO: 2 (i.e., 1A1), SEQ ID NO: 3 and SEQ ID NO: 4 (i.e., 1F8), SEQ ID NO: 5 and SEQ ID NO: 6 (i.e., 2A11), SEQ ID NO: 7 and SEQ ID NO: 8 (i.e., 2C2), SEQ ID NO: 9 and SEQ ID NO:10 (i.e., 2D7), SEQ ID NO: 11 and SEQ ID NO: 12 (i.e., 2G4), SEQ ID NO: 13 and SEQ ID NO: 14 (i.e., 2G11), SEQ ID NO: 15 and SEQ ID NO: 16 (i.e., 6F4), SEQ ID NO: 17 and SEQ ID NO: 18 (i.e., 5H1), SEQ ID NO: 19 and SEQ ID NO: 20 (i.e., 5F6), SEQ ID NO: 21 and SEQ ID NO: 22 (i.e., 1F3), SEQ ID NO: 23 and SEQ ID NO: 24 (i.e., 2A4), SEQ ID NO: 25 and SEQ ID NO: 26 (i.e., 2B12), SEQ ID NO: 27 and SEQ ID NO: 28 (i.e., 13A11), SEQ ID NO: 29 and SEQ ID NO: 30 (i.e., 15E1), SEQ ID NO: 31 and SEQ ID NO: 32 (i.e., 13H3), SEQ ID NO: 33 and SEQ ID NO: 34 (i.e., 14A8), SEQ ID NO: 35 and SEQ ID NO: 36 (i.e., 16H3), SEQ ID NO: 37 and SEQ ID NO: 38 (i.e., 1A1), SEQ ID NO: 39 and SEQ ID NO: 40 (i.e., 1A1-A), SEQ ID NO: 41 and SEQ ID NO: 42 (i.e., 1A1-Q), SEQ ID NO: 43 and SEQ ID NO: 44 (i.e., 1A2), SEQ ID NO: 45 and SEQ ID NO: 46 (i.e., 1A8), SEQ ID NO: 47 and SEQ ID NO: 48 (i.e., 1B1), SEQ ID NO: 49 and SEQ ID NO: 50 (i.e., 1B2), SEQ ID NO: 51 and SEQ ID NO: 52 (i.e., 1H3), SEQ ID NO: 53 and SEQ ID NO: 54 (i.e., 1H3-Q), SEQ ID NO: 55 and SEQ ID NO: 56 (i.e., 1H3-A), SEQ ID NO: 57 and SEQ ID NO: 58 (i.e., 2A2), SEQ ID NO: 59 and SEQ ID NO: 60 (i.e., 2A3), SEQ ID NO: 61 and SEQ ID NO: 62 (i.e., 2A6), SEQ ID NO: 63 and SEQ ID NO: 64 (i.e., 2A10), SEQ ID NO: 65 and SEQ ID NO: 66 (i.e., 2B1), SEQ ID NO: 67 and SEQ ID NO: 68 (i.e., 2C6), SEQ ID NO: 69 and SEQ ID NO: 70 (i.e., 2E7), SEQ ID NO: 71 and SEQ ID NO: 72 (i.e., 2E9), SEQ ID NO: 73 and SEQ ID NO: 74 (i.e., 2F1), SEQ ID NO: 75 and SEQ ID NO: 76 (i.e., 2F3), SEQ ID NO: 77 and SEQ ID NO: 78 (i.e., 34C5), SEQ ID NO: 79 and SEQ ID NO: 80 (i.e., A1A), and SEQ ID NO: 81 and SEQ ID NO: 82 (i.e., T4J).

In some particular examples, a CD47 antibody suitable for the compositions of this invention would include a combined VH/VL chain sequence that is selected from the group consisting of SEQ ID NO: 3 and SEQ ID NO: 4 (i.e., 1F8), SEQ ID NO: 31 and SEQ ID NO: 32 (i.e., 13H3), or SEQ ID NO: 79 and SEQ ID NO: 80 (i.e., A1A). Antibodies 13H3 and A1A are the affinity matured clones of antibody 1F8. And the amino acid sequences of the three antibodies are highly similar.

### Example 1: Isolation of Mononuclear Cells from Human Peripheral Blood

Human peripheral blood mononuclear cells (PBMCs) were isolated by Ficoll-Hypaque density gradient centrifugation. All experiments with human blood were conducted under Institutional Review Board (IRB) approved protocols.

Fresh blood sample was diluted with phosphate-buffered saline (PBS), and then carefully layered on top of Ficoll-Paque Plus density gradient centrifugation media. The sample was then centrifuged at 2000 rpm for 20 min with breaks off. After density gradient centrifugation, differential migration of cells during centrifugation resulted in the formation of layers containing different cell types. PBMCs can be found together with other low-density slowly sedimenting particles (e.g., platelets) at the interface between the plasma and the Ficoll-Paque layer. The PBMCs were harvested by pouring the top layer, transferred to a new tube and then washed with PBS. Through centrifugation at 1500 rpm for 10 min, freshly isolated PBMCs were collected and re-suspended in PBS for further T cell subset isolation.

### Example 2: Generation of Macrophages from Human Peripheral Blood CD14⁺ Monocytes

Human peripheral blood CD14⁺ monocytes were isolated by a positive selection method using a magnetic activated cell sorting (MACS) system according to the manufacturer's protocol.

Then, the monocytes isolated by MACS were subsequently cultured in fresh complete medium supplemented with recombinant human granulocyte-macrophage colony-stimulating factor GM-CSF (50 ng/ml) and human recombinant IL-4 (35 ng/ml) to activate differentiated macrophages. After 6 days of culture (on day 7), cells were harvested, pooled together and counted for next use.

### Example 3. Macrophage Viability Assay

The cells from Example 2 were plated into a 96-well flat bottom plate at a density of 0.05×10⁶ cells/well. After 2 hours of incubation, the macrophages were well attached to the plate. Then selected small molecule therapeutic agents were added to the well and cocultured for 16 hours. On day 8, macrophage viability was assessed by CellTiter-Glo® Luminescent Cell Viability Assay, according to the manufacture's instruction.

TABLE 1
Selected small molecule therapeutic agents (Vendors are provided)

| Therapeutic | Mechanism | Vendor & |
|---|---|---|
| agents | of Action | Catalog Number |
| Lenalidomide | Immunomodulatory, po | MedChemExpress (HY-A0003) |
| Bendamustine | Alkylating agent, iv | MedChemExpress (HY-B0077) |
| Ibrutinib | BTK inhibitor, po | MedChemExpress (HY-10997) |
| Venetoclax | Bcl-2 inhibitor, po | MedChemExpress (HY-15531) |
| Copanlisib | PI3K inhibitor, po | MedChemExpress (HY-15346) |
| Duvelisib | PI3K inhibitor, po | MedChemExpress (HY-17044) |
| Idelalisib | PI3K inhibitor, po | MedChemExpress (HY-13026) |
| Azacitidine | Nucleoside | MedChemExpress (HY-10586) |
| | antimetabolite, DNA | |
| | Methyltransferse | |

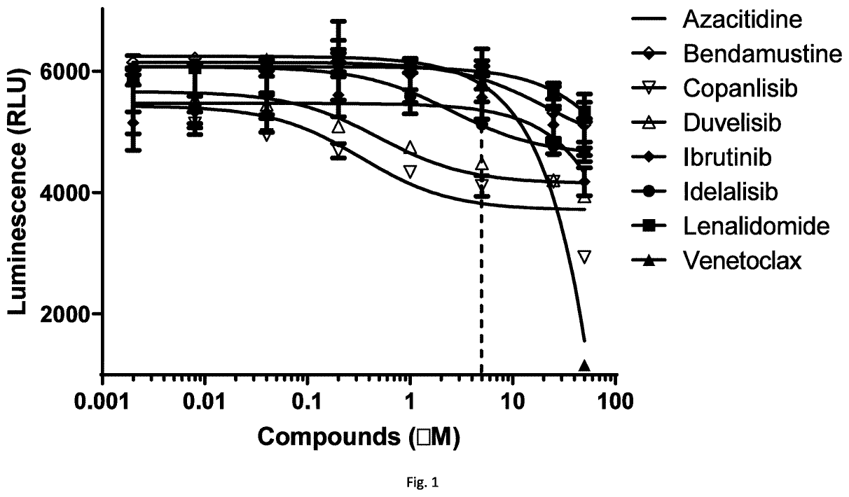

Results showed that therapeutic agents Copanlisib, Duvelisib and Idelalisib present obvious toxicity to macrophages at a dose of 0.1-50 μM. Azacytidine, Bendamustine, lbrutinib, Lenalidomide and Venetoclax showed little toxic to macrophages when dose is lower than 10 μM, although it showed toxicity to macrophages when dose is as high as 50 μM (see FIG. 1, Table 2).

TABLE 2
Results of Macrophage Viability Assay

| | Cpd(uM) | | | | | | | |
|---|---|---|---|---|---|---|---|---|
| Cpd | 50 | 25 | 5 | 1 | 0.2 | 0.04 | 0.008 | 0.002 |
| Azacitidine | 4580 | 4560 | 4768 | 5140 | 5160 | 4792 | 4648 | 4076 |
| | 5420 | 5520 | 6132 | 6108 | 6136 | 5892 | 6100 | 6020 |
| | 4932 | 5324 | 6032 | 6256 | 6440 | 6380 | 6132 | 5860 |
| Bendamustine | 4112 | 4340 | 5064 | 5436 | 5768 | 5788 | 6028 | 5908 |
| | 5500 | 5884 | 6424 | 6220 | 6320 | 6248 | 6248 | 6292 |
| | 5736 | 5724 | 6308 | 6200 | 6664 | 6124 | 6356 | 6220 |
| Copanlisib | 2748 | 4108 | 3780 | 4156 | 4572 | 4856 | 5272 | 5856 |
| | 3032 | 4176 | 4352 | 4492 | 4572 | 5120 | 4772 | 5928 |
| | 3028 | 4248 | 4196 | 4368 | 4928 | 4872 | 5388 | 6000 |
| Duvelisib | 3972 | 4176 | 4464 | 4592 | 5028 | 5228 | 5484 | 5688 |
| | 3992 | 4276 | 4584 | 4776 | 4984 | 5348 | 5444 | 5876 |
| | 3860 | 4156 | 4404 | 4880 | 5264 | 5760 | 5664 | 6184 |
| Ibrutinib | 3732 | 4680 | 4864 | 5056 | 4932 | 4688 | 4880 | 4948 |
| | 4296 | 5100 | 5828 | 5624 | 5736 | 5488 | 5316 | 4988 |
| | 4508 | 5572 | 6004 | 6224 | 6152 | 5780 | 5780 | 5520 |
| Idelalisib | 4404 | 4508 | 5092 | 5376 | 6040 | 6144 | 6164 | 5888 |
| | 4648 | 4884 | 5220 | 5640 | 6048 | 6304 | 6056 | 5908 |
| | 4964 | 4908 | 5060 | 5744 | 6220 | 5916 | 5948 | 5980 |
| Lenalidomide | 5208 | 5432 | 5840 | 6016 | 6216 | 6152 | 5992 | 5972 |
| | 5124 | 5752 | 6236 | 6108 | 6088 | 6132 | 5996 | 5872 |
| | 5644 | 5744 | 6108 | 6088 | 6456 | 5892 | 6180 | 5776 |
| Venetoclax | 1240 | 4696 | 5760 | 6052 | 7260 | 6288 | 6148 | 5968 |
| | 1132 | 4596 | 5660 | 6020 | 6072 | 6072 | 6172 | 6024 |
| | 1108 | 4776 | 6120 | 6088 | 5756 | 6264 | 5912 | 5856 |

### Example 4: Fluorescence-Activated Cell Sorting (FACS) Analysis of Expression Levels of CD47 and Calreticulin on Tumor Cell Surface

For FACS analysis, on the day of analysis (day 1), Toledo cells were seeded into 96 well plate and cultured at a density of 0.2×10⁶ cells/well. Then the cells were treated with the selected small molecule therapeutic agents for 12 hours.

On day 2, cells were harvested, washed twice and then re-suspended in 100 uL FACS buffer subject to FACS analysis. For detection of the expression of calreticulin and CD47, cells were incubated with anti-calreticulin antibody and commercially available anti-CD47 antibody (BD Biosciences, Catalog number: 561249) in dark at 4° C. for 30 mins.

Then, the cells were subject to FACS analysis. Data was acquired and analyzed.

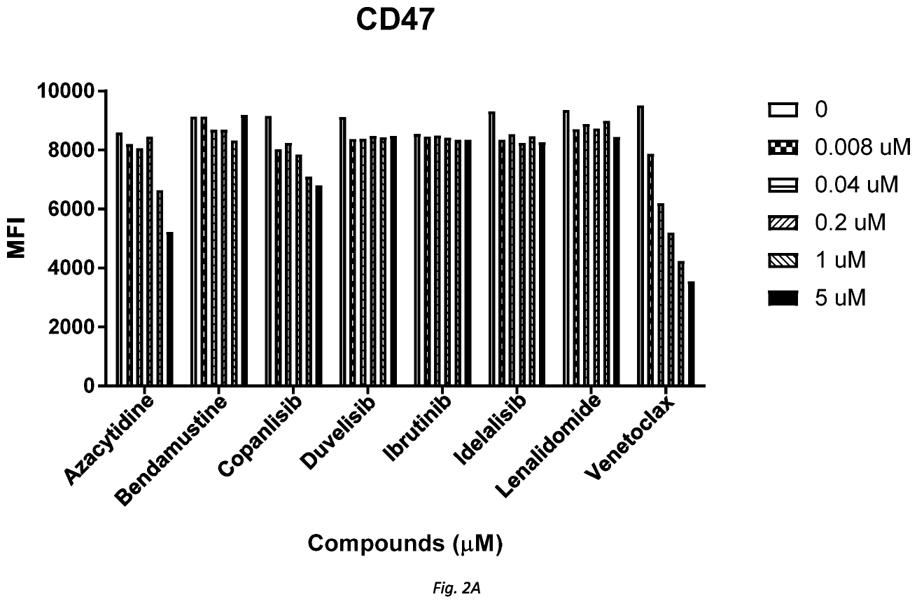

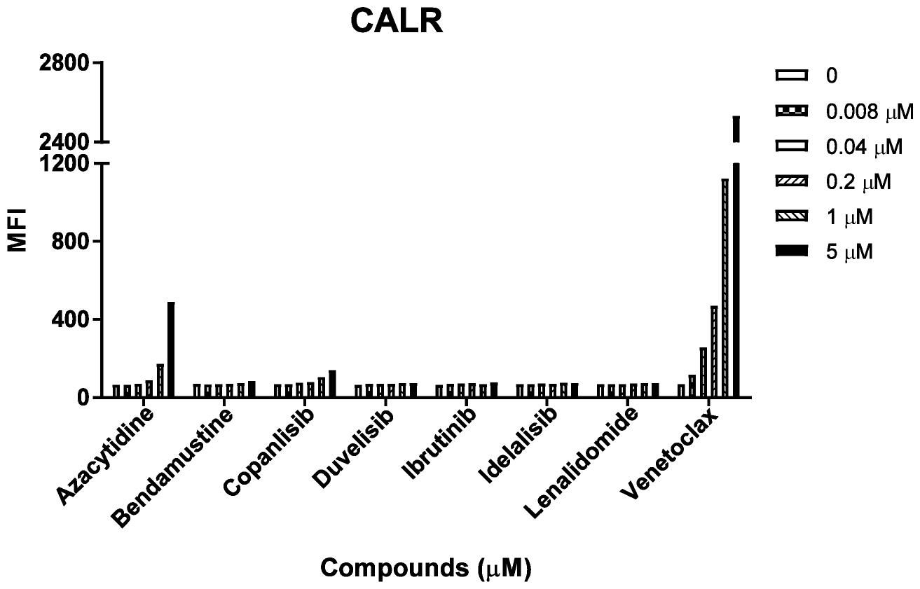

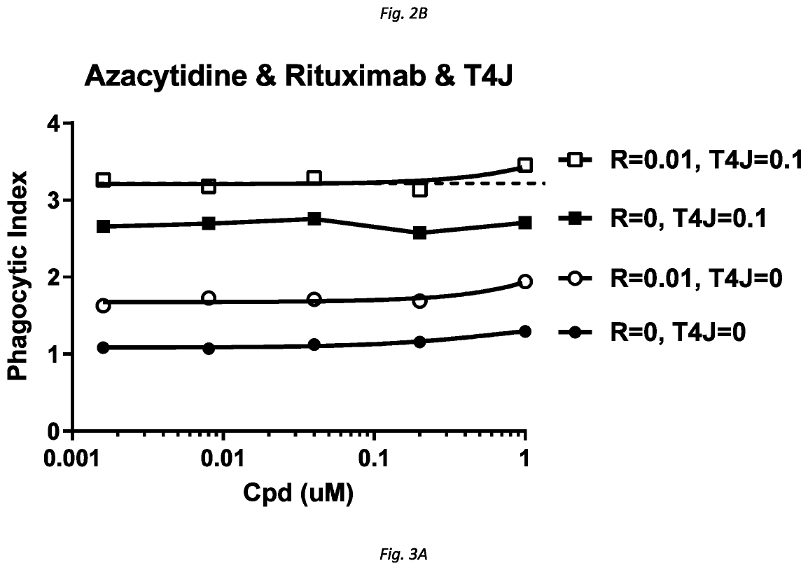

In this assay, we studied the effects of the selected small molecule therapeutic agents on the expression level of calreticulin and CD47, to see if the therapeutic agents had the capability to affect the phagocytic ability of macrophage. Results showed that Azacytidine and Venecoclax can obviously inhibit CD47 expression level by inhibiting almost 50% of CD47 expression at a dose of 5 μM, while Copanlisib only slightly inhibited CD47 expression level. Results also showed that Azacytidine and Venecoclax can obviously increase the expression level of calreticulin to about 20 folds and 120 folds at a dose of 5 μM, respectively, while Copanlisib only slightly increased the expression level of Calreticulin (See FIG. 2, Table 3).

TABLE 3
The expression level of CD47 and calreticulin on tumor cell analyzed by FACS

| | Mean Fluorescence Intensity (MFI) | | | | | | Mean Fluorescence Intensity (MFI) | | | | | |
|---|---|---|---|---|---|---|---|---|---|---|---|---|
| | CD47 | | | | | | Calreticulin | | | | | |
| | | 0.008 | 0.04 | 0.2 | 1 | 5 | | 0.008 | 0.04 | 0.2 | 1 | 5 |
| | 0 | uM | uM | uM | uM | uM | 0 | uM | uM | uM | uM | uM |
| Azacytidine | 8590 | 8204 | 8062 | 8460 | 6638 | 5229 | 66.8 | 65.8 | 71.9 | 88.4 | 174 | 491 |
| Bendamustine | 9138 | 9132 | 8684 | 8687 | 8319 | 9192 | 70.2 | 68.6 | 70.1 | 70.2 | 76.3 | 86.2 |
| Copanlisib | 9158 | 8024 | 8243 | 7844 | 7097 | 6803 | 69.2 | 69.8 | 77 | 79.4 | 106 | 142 |
| Duvelisib | 9124 | 8368 | 8376 | 8471 | 8432 | 8482 | 65.7 | 71.4 | 71.2 | 71.4 | 75.7 | 75.9 |
| Ibrutinib | 8544 | 8453 | 8490 | 8415 | 8345 | 8345 | 65.2 | 70.6 | 73.1 | 74.5 | 69.9 | 79 |
| Idelalisib | 9310 | 8342 | 8538 | 8246 | 8465 | 8263 | 69.3 | 69.4 | 72.9 | 70.6 | 77.6 | 75.8 |
| Lenalidomide | 9360 | 8706 | 8881 | 8730 | 8985 | 8443 | 69 | 69.3 | 70.1 | 73.4 | 75.6 | 75.2 |
| Venetoclax | 9512 | 7873 | 6201 | 5206 | 4232 | 3540 | 70 | 117 | 258 | 471 | 1123 | 2533 |

### Example 5: Analysis of Phagocytic Ability of Macrophages by FACS

FACS-based phagocytosis assays were performed to evaluate the phagocytic abilities of macrophages.

Toledo Cells (a tumor cell line, as a target) were labelled with CellTrace™ Far Red and plated into 96-well plate at a density of 0.15×10⁶ cells/well for 12 hours. The Macrophages were prepared as discussed above and then labelled with CellTrace Violet. Next, the macrophages were added into the plate as an effector at an effector:target (Toledo) ratio of 1:3, giving a final density of macrophages at 0.05×10⁶ cells/well. Macrophages and tumor cells were cocultured for 2-6 h at 37° C. in the presence of the indicated treatments (small molecule therapeutic agents, CD47 antibody, CD20 antibody or combinations). The incubation time depending on the phagocytosis rate, expecting phagocytosis of 5-20% cells.

Then, cells were acquired and data were analyzed.

Results demonstrated that the isolated anti-CD47 antibody (i.e., T4J) or Rituximab itself can promote macrophage phagocytosis, moreover, combination of the isolated anti-CD47 antibody (i.e., T4J) and Rituximab enhances the phagocytic ability of macrophages even higher (See Table 4).

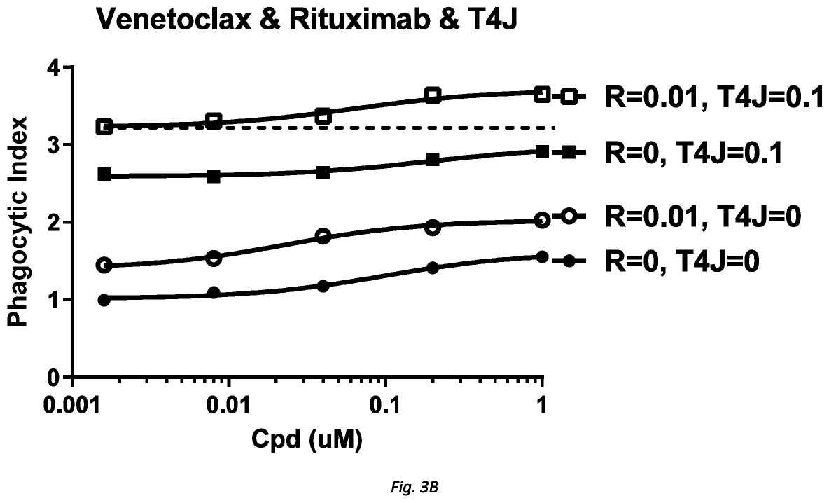

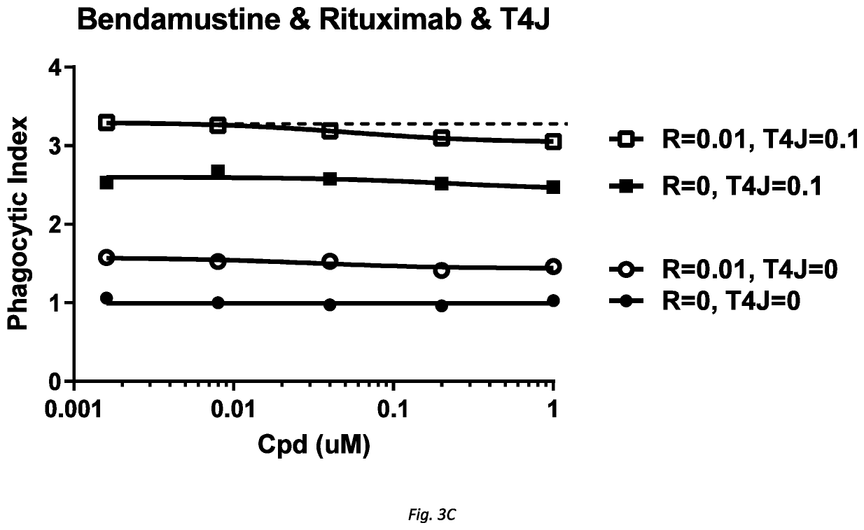

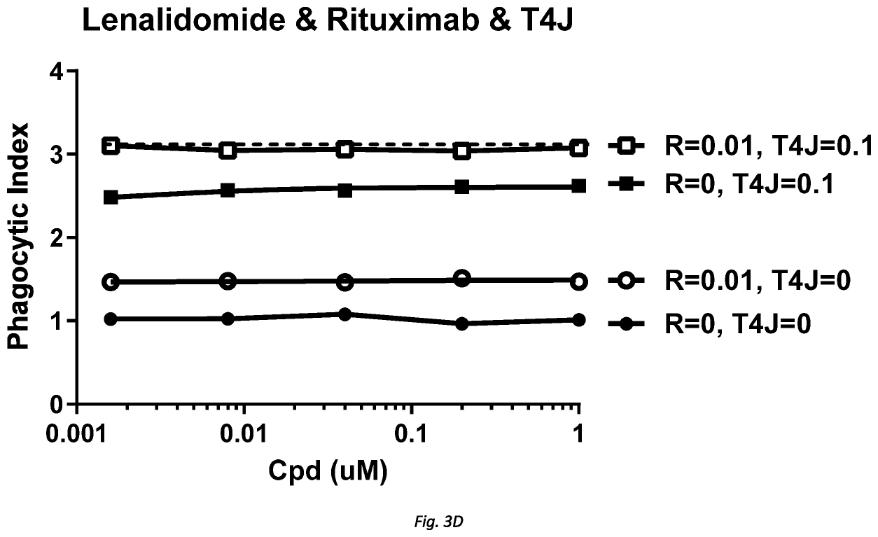

Further, Axacytidine or Venetoclax itself can also enhance the phagocytic ability of macrophages, moreover, the triple combination (isolated anti-CD47 antibody (i.e., T4J), Rituximab and Axacytidine or Venetoclax) enhances the phagocytic ability of macrophages much higher. On the other hands, Bendamustine or Lenalidomide does not show the synergistic effects (see FIG. 3, Table 4).

TABLE 4
Treatment effects of triple combinationtherapy on macrophage phagocytosis

| | Azacitidine | | | | Venetoclax | | | |
|---|---|---|---|---|---|---|---|---|
| | T4J = 0 | | T4J = 0.1 ug/ml | | T4J = 0 | | T4J = 0.1 ug/ml | |
| Cpd (uM) | R = 0 | R = 0.01 ug/ml | R = 0 | R = 0.01 ug/ml | R = 0 | R = 0.01 ug/ml | R = 0 | R = 0.01 ug/ml |
| 0 | 1.00 | 1.57 | 2.41 | 3.00 | 1.00 | 1.38 | 2.30 | 2.89 |
| 0.0016 | 1.09 | 1.63 | 2.66 | 3.26 | 1.00 | 1.45 | 2.62 | 3.23 |
| 0.008 | 1.07 | 1.73 | 2.69 | 3.18 | 1.10 | 1.54 | 2.59 | 3.30 |
| 0.04 | 1.12 | 1.71 | 2.76 | 3.29 | 1.17 | 1.82 | 2.64 | 3.37 |
| 0.2 | 1.16 | 1.69 | 2.57 | 3.13 | 1.41 | 1.93 | 2.81 | 3.64 |
| 1 | 1.30 | 1.94 | 2.70 | 3.45 | 1.56 | 2.03 | 2.91 | 3.65 |
| | Bendamustine | | | | Lenalidomide | | | |
| | T4J = 0 | | T4J = 0.1 ug/ml | | T4J = 0 | | T4J = 0.1 ug/ml | |
| Cpd (uM) | R = 0 | R = 0.01 ug/ml | R = 0 | R = 0.01 ug/ml | R = 0 | R = 0.01 ug/ml | R = 0 | R = 0.01 ug/ml |
| 0 | 1.00 | 1.36 | 2.20 | 3.03 | 1.00 | 1.31 | 2.20 | 2.86 |
| 0.0016 | 1.06 | 1.58 | 2.53 | 3.29 | 1.02 | 1.46 | 2.48 | 3.10 |
| 0.008 | 1.00 | 1.52 | 2.67 | 3.26 | 1.03 | 1.47 | 2.57 | 3.04 |
| 0.04 | 0.98 | 1.53 | 2.57 | 3.19 | 1.08 | 1.46 | 2.56 | 3.06 |
| 0.2 | 0.96 | 1.41 | 2.51 | 3.10 | 0.96 | 1.51 | 2.61 | 3.04 |
| 1 | 1.03 | 1.46 | 2.47 | 3.05 | 1.01 | 1.47 | 2.62 | 3.07 |

### Example 6 In Vivo Test of the Anti-Tumor Efficacy of Anti-CD47 Antibody in Combination with Anti-CD20 Antibody in a Tumor Xenograft Model

All animal experimental procedures were approved by the Institutional Animal Care and Use Committee (IACUC). Anti-CD47 antibody (i.e. T4J) and anti-CD20 antibody (i.e. rituximab) were used in this study.

To establish the xenograft model, human diffuse large B Cell lymphoma (WSU-DLCL2 cells) were subcutaneously (s.c.) injected into 6- to 7-week-old NOD/SCID mice (Shanghai Lingchang Biotechnology Co., Ltd, Shanghai, China) for tumor development. When tumor volume reaches a mean value of approximately 94 mm³, 32 mice were divided into 4 groups of 8 equally and injected intravenously (i.v.) with PBS (control group), anti-CD47 antibody (i.e. T4J), rituximab, or anti-CD47 antibody T4J+ rituximab at a dose of 5 mg/kg, respectively (See, Table 5). The anti-tumor efficacy studies were performed using a twice per week dosing schedule (5 mg/kg) for 4 weeks.

TABLE 5
Description of the treatment groups and control group

| | No. | | | Dose | Routeof | |
|---|---|---|---|---|---|---|
| Group | of | | Dosage | Vol. | adminis- | Dosing |
| No. | Mice | Treatment | (mg/kg) | (ml/kg) | tration | Schedule |
| 1 | 8 | PBS(control) | 0 | 5 | i.v. | BIW × 4 |
| | | | | | | weeks |
| 2 | 8 | Rituximab | 5 | 5 | i.v. | BIW × 4 |
| | | | | | | weeks |
| 3 | 8 | T4J | 5 | 5 | i.v. | BIW × 4 |
| | | | | | | weeks |
| 4 | 8 | Rituximab | 5 | 5 | i.v. | BIW × 4 |
| | | | | | | weeks |
| | | T4J | 5 | 5 | i.v. | BIW × 4 |
| | | | | | | weeks |

The first day of antibody administration was designated as day 0. Mice were then monitored for tumor development and progression, and observation continued until day 43. Tumor volumes were measured twice a week in two dimensions (length and width) using a caliper, and the volumes were calculated using the following formula: Volume (V)=(L×W×W)/2, where L is tumor length (the longest tumor dimension) and W is tumor width (the longest tumor dimension perpendicular to L). The percentage of tumor growth inhibition (TGI) was calculated as follows: 100%×(1−[(V_{treated}(^{finalday})−V_{treated}(^{initial day}))/(V_{control}(^{final day})−V_{control}(^{initial day})))]), where V is the tumor volume. Tumor body weight was measured twice per week after randomization and at day 43. Antibody treatment was then stopped after 43 days of study and mice were euthanized and necropsied for evidence of tumors.

Group comparisons were carried out using one-way analysis of variance (ANOVA). Data analysis were performed using IBM SPSS software version 18.0 (IBM, Armonk, N.Y., U.S.). Values of tumor volume and tumor body weight were expressed as mean±standard error of the mean (SEM). A P value of less than 0.05 (p<0.05) was considered statistically significant.

After four weeks of dosing, the group treated with T4J showed no anti-tumor efficacy (1% TGI, p=1.000) compared with the control group. The group treated with a combination of T4J (5 mg/kg) and Rituximab (5 mg/kg) showed significant anti-tumor efficacy compared with the control group (63% TGI, p=0.004), and demonstrated significantly improved anti-tumor efficacy (p=0.002) compared with the group treated with T4J, and showed improved but not significant anti-tumor efficacy (p=0.086) compared with the group treated with rituximab (Table 6). No deaths or adverse effect occurred in all groups during the period of study. Therefore, results indicate that rituximab can increase the therapeutic effects of T4J in the tumor xenograft model.

TABLE 6
Anti-tumor efficacy of antibody

| | | Tumor Volume | | | P value | P value |
|---|---|---|---|---|---|---|
| | | (mm³) on day 43 | TGI | T/C | (compared | (compared |
| Group | Treatment | (Mean ± SEM) | (%) | (%) | with control) | with group 4) |
| 1 | PBS (Control) | 2161.32 ± 252.88 | — | — | — | |
| 2 | Rituximab 5 mg/kg | 1578.37 ± 241.53 | 27 | 73 | 0.376 | 0.086 |
| 3 | T4J 5 mg/kg | 2148.89 ± 227.87 | 1 | 99 | 1.000 | 0.002 |
| 4 | Rituximab + T4J | 794.9 ± 174.05 | 63 | 37 | 0.004 | |
| | 5 mg/kg + 5 mg/kg | | | | | |

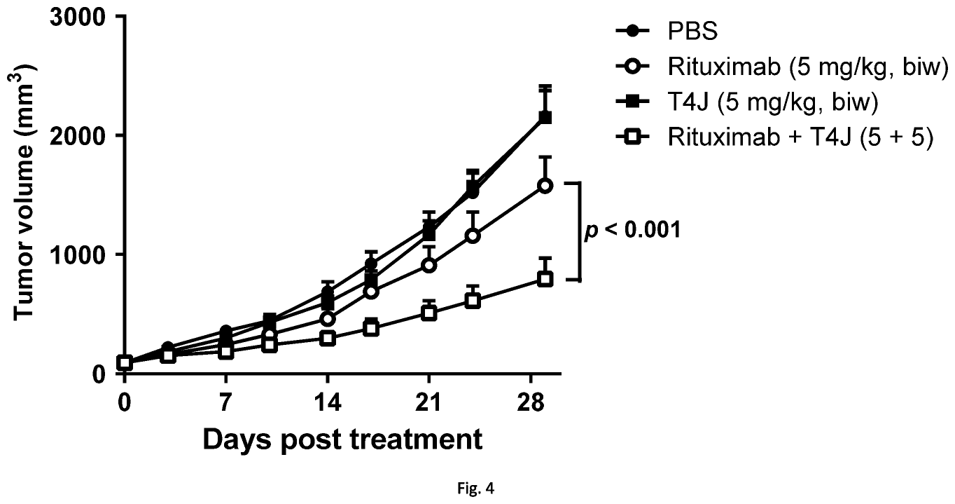

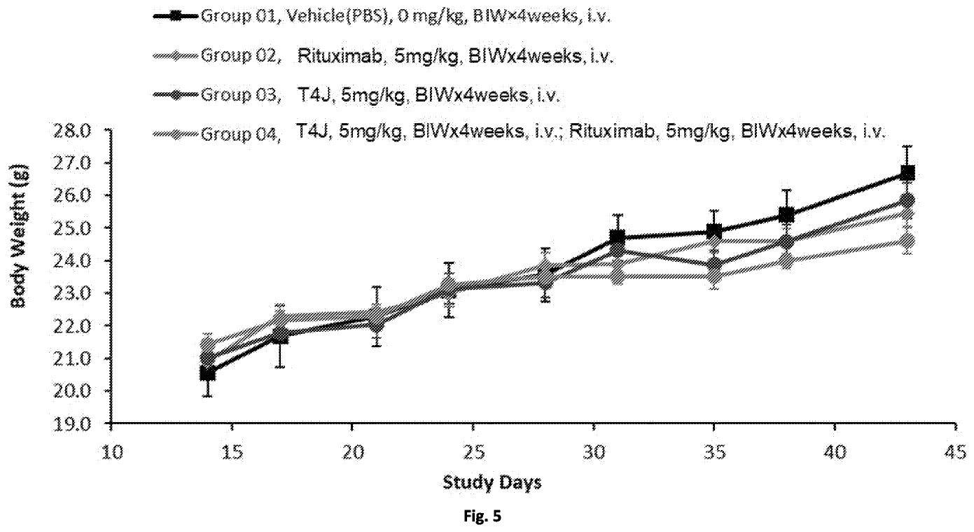

Analysis of tumor growth showed that the group treated with T4J combined with rituximab showed significant tumor regression at day 43 (See FIGS. 4-5, Table 7). In summary, the group treated with a combination of T4J and rituximab improved therapeutic efficacy compared with the group treated with any of single agents, either T4J or rituximab itself (See FIGS. 4-5, Table 7).

TABLE 7
Analysis of Tumor Body Weight

| | | Tumor Body | | | P value | P value |
|---|---|---|---|---|---|---|
| | | Weight (mg) on day 43 | TGI | T/C | (compared | (compared |
| Group | Treatment | (Mean ± SEM) | (%) | (%) | with control) | with group 4) |
| 1 | PBS (Control) | 2520.7 ± 354.9 | — | — | — | |
| 2 | Rituximab 5 mg/kg | 1673.7 ± 269.0 | 34 | 66 | 0.174 | 0.365 |
| 3 | T4J 5 mg/kg | 2426.0 ± 283.0 | 4 | 96 | 0.995 | 0.007 |
| 4 | Rituximab + T4J5 mg/kg + 5 mg/kg | 1009.0 ± 207.7 | 60 | 40 | 0.004 | |

### Example 7 In Vivo Test of Anti-Tumor Efficacy of Anti-CD47 Antibody in Combination with Azacitidine (AZA) in a Tumor Xenograft Model

All animal experimental procedures were approved by the Institutional Animal Care and Use Committee (IACUC). In this study, therapeutic efficacy of anti-CD47 antibody (i.e. T4J) and AZA alone or jointly in the treatment of the HL-60 xenograft model was evaluated.

### Cell Culture

HL-60 cells (a human promyelocytic leukemia cell line, ATCC® cat #CCL-240) were maintained in suspension culture in complete cell growth medium at 37° C. in an atmosphere with 5% CO₂. The cells were split twice a week to maintain an exponential growth. After culturing, cells were harvested and counted for tumor inoculation.

### Tumor Inoculation and Animal Grouping

To establish the xenograft model, HL-60 cells (10×10⁶) were suspended in 0.2 mL of PBS with the same volume of Matrigel and subcutaneously injected into the right flank of each mouse. When tumor volume reaches a mean value of approximately 72 mm³, i.e., on day 6 after inoculation, mice were divided into several groups and treated with PBS (control group), anti-CD47 antibody (i.e., T4J), AZA, or a combination of T4J and AZA, respectively.

### Observations

At the time of routine monitoring, the animals were daily checked for any effects of tumor growth and treatments on normal behavior such as mobility, food and water consumption (by looking only), body weight gain/loss (body weights were measured twice a week), eye/hair matting and any other abnormal effects. Death and observed clinical signs were recorded.

### Tumor Measurements

Tumor size was measured twice a week in two dimensions using a caliper, and the volume was calculated using the formula: Volume (V)=(L×W×W)/2, where L is tumor length (the longest tumor dimension) and W is tumor width (the longest tumor dimension perpendicular to L). The tumor volume was then used for calculating T/C values. The T/C value (in percentage) is an indicator of antitumor effectiveness, in which T and C are the mean volumes of the treatment group and the control groups, respectively.

The percentage of tumor growth inhibition (TGI) was calculated for each group as follows: 100%×(1−[(V_{treated}(^{final day})−V_{treated}(^{initial day}))/V_{control}(^{final day})−V_{control}(^{initial day})))], where V is the tumor volume.

Tumor body weight was measured at the end of the study. T/C weight value (in percentage) was calculated using the formula: T/C weight %=T weight/C weight×100% where T weight and C weight were the mean tumor body weight of the treatment group and the control group (vehicle group), respectively.

Treatment efficacy was evaluated using Jin's formula:

Q=TGI(A+B))/(TGI(A)+TGI(B)−TGI(A)×TGI(B))

According to Jin's formula, Q<0.85 indicates antagonistic effect, 0.85≤Q<1.15 indicates additive effect, and Q≥1.15 indicates synergistic effect.

### Statistical Analysis

T-test was performed to compare tumor body weight among groups. One-way ANOVA was performed to compare tumor volume among groups, and when a significant F-statistics (a ratio of treatment variance to the error variance) was obtained, comparisons between groups were carried out with Games-Howell test. All data were analyzed using IBM SPSS software. A P value of less than 0.05 (p<0.05) was considered statistically significant.

### Results:

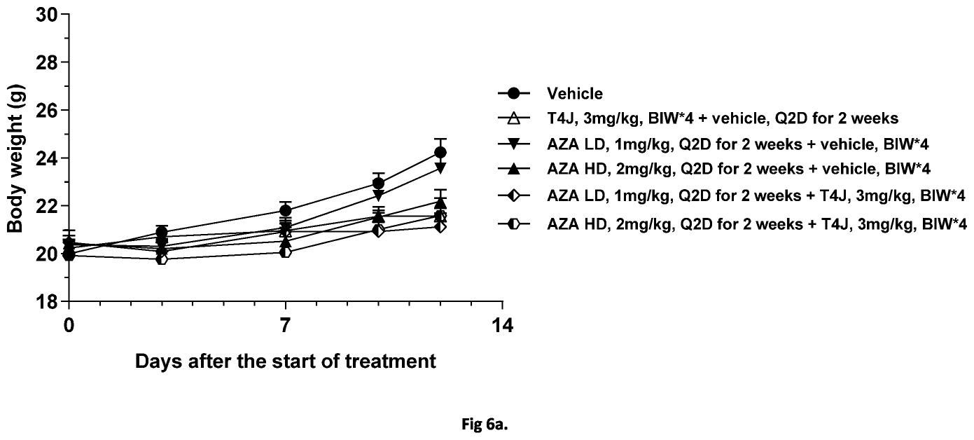

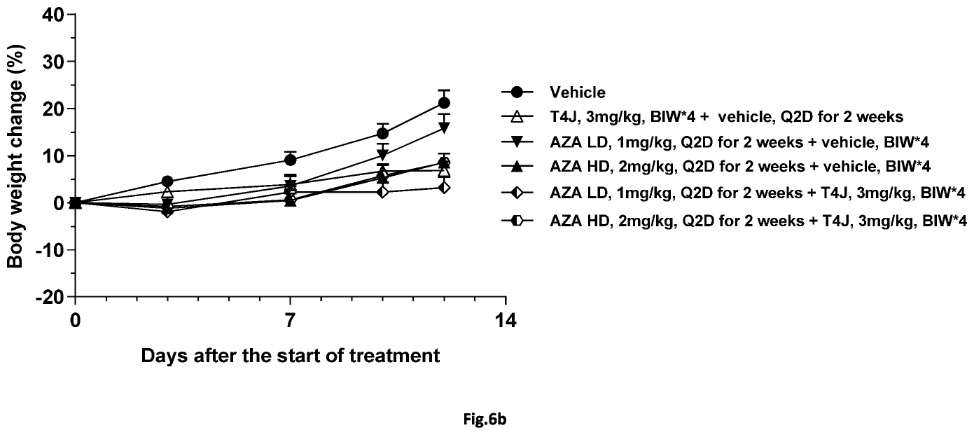

Tumor body weight was monitored regularly as an indirect measure of toxicity. No deaths or adverse effect occurred in all groups during the period of study. Tumor body weight changes in different treatment groups are shown in FIGS. 6a and 6b.

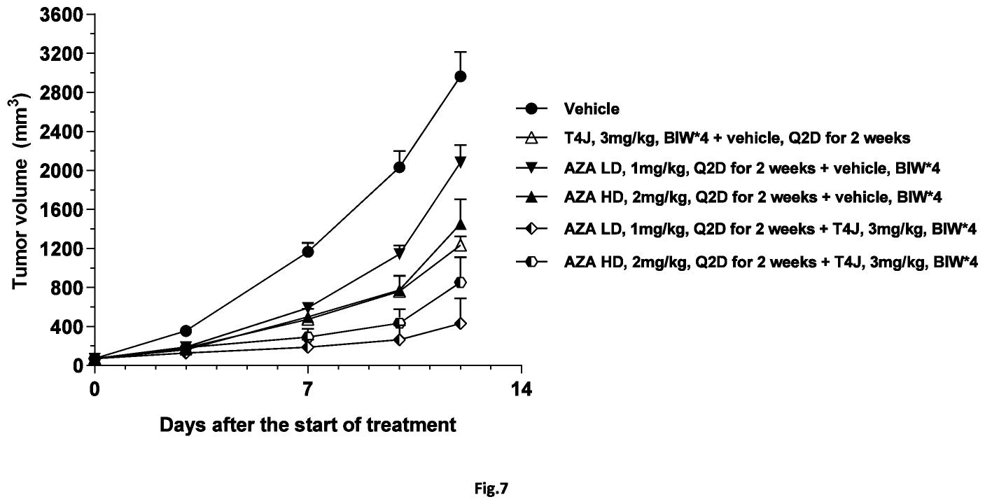

Tumor volume over time was shown in Table 8. Tumor growth curve was shown in FIG. 7.

TABLE 8
Tumor volume over time

| | Tumor volume (mm³)^{a} | | | | | |
|---|---|---|---|---|---|---|
| | | T4J | AZA | AZA | T4J + AZA | T4J + AZA |
| Days | Vehicle | 3 mg/kg | 1 mg/kg | 2 mg/kg | 3 + 1 mg/kg | 3 + 2 mg/kg |
| 0^{b} | 70 ± 4 | 70 ± 7 | 70 ± 7 | 70 ± 7 | 70 ± 6 | 70 ± 7 |
| 3 | 355 ± 31 | 180 ± 10 | 189 ± 11 | 161 ± 20 | 127 ± 20 | 184 ± 29 |
| 7 | 1166 ± 90 | 473 ± 36 | 589 ± 41 | 498 ± 82 | 186 ± 77 | 289 ± 85 |
| 10 | 2033 ± 166 | 760 ± 52 | 1142 ± 86 | 773 ± 145 | 262 ± 147 | 434 ± 140 |
| 12 | 2964 ± 248 | 1235 ± 89 | 2081 ± 177 | 1452 ± 253 | 431 ± 254 | 850 ± 258 |

^{a}Mean ± SEM;
^{b}days after first dosing

### CONCLUSION

As summarized in Table 9, the group treated with 1 mg/kg AZA showed no obvious antitumor activity with a mean volume of 2081±177 mm³ by comparing with the control group (2964±248 mm³). The group treated with 3 mg/kg TJC4 and 1 mg/kg AZA for 12 days showed a significant decrease in tumor volume (431±254 mm³) comparing to the control group (2964±248 mm³), indicating a significant anti-tumor effect.

The group treated with 2 mg/ml AZA showed certain anti-tumor activity with a mean volume of 1452±253 mm³. The group treated with 3 mg/kg TJC4 and 2 mg/kg AZA for 12 days showed a significant decrease in tumor volume (850±258 mm³) comparing to the control group (2964±248 mm³), indicating a significant antitumor effect.

Statistical evaluation of two combination treatment groups was conducted based on the JIN's Formulation. Results showed that combination treatment with 1 mg/kg AZA and 3 mg/kg TJC4 resulted in Q value of 1.22, demonstrating a synergistic effect between AZA and CD47 antibody; and treatment with 2 mg/kg AZA and 3 mg/kg TJC4 resulted in Q value of 0.90, demonstrating an additive effect between AZA and CD47 antibody.

TABLE 9
Antitumor efficacy of antibody T4J
in combination with AZAat day 12

| | Tumor Size | | | | | Drug |
|---|---|---|---|---|---|---|
| | (mm³)^{a} | T/C | TGI | p | Q | combina- |
| Treatment | at day 12 | (%) | (%) | value^{b} | value | tion |
| Vehicle | 2964 ± 248 | — | — | — | — | — |
| T4J(3 mg/kg) | 1235 ± 89 | 41.68 | 59.74 | <0.001 | — | — |
| AZA (1 mg/kg) | 2081 ± 177 | 70.21 | 30.52 | 0.032 | — | — |
| AZA (2 mg/kg) | 1452 ± 253 | 48.98 | 52.27 | <0.001 | — | — |
| AZA + T4J(1 + | 431 ± 254 | 14.53 | 87.55 | <0.001 | 1.22 | synergistic |
| 3 mg/kg) | | | | | | effect |
| AZA + T4J(2 + | 850 ± 258 | 28.68 | 73.04 | <0.001 | 0.90 | Additive |
| 3 mg/kg) | | | | | | Effect |

^{a}Mean ± SEM.
^{b}p value is calculated based on tumor size.

What is claimed is:

1. A pharmaceutical composition comprising an isolated antibody or an immunologically active fragment thereof that specifically binds human CD47, a therapeutic agent, and a pharmaceutically acceptable carrier; wherein the isolated antibody or an immunologically active fragment thereof that specifically binds human CD47 comprises a heavy chain variable region (VH) having amino acid sequence that is at least 95% identical to an amino acid sequence selected from the group consisting of SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, SEQ ID NO: 67, SEQ ID NO: 69, SEQ ID NO: 71, SEQ ID NO: 73, SEQ ID NO: 75, SEQ ID NO: 77, SEQ ID NO: 79, and SEQ ID NO: 81; and a light chain variable region (VL) having amino acid sequence that is at least 95% identical to an amino acid sequence selected from the group consisting of SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 70, SEQ ID NO: 72, SEQ ID NO: 74, SEQ ID NO: 76, SEQ ID NO: 78, SEQ ID NO: 80, and SEQ ID NO: 82.

2. The pharmaceutical composition of claim 1, wherein the isolated antibody or an immunologically active fragment thereof that specifically binds human CD47 comprises a VH/VL pair, wherein the VH/VL pair comprises VH and VL chain sequences selected from the group consisting of SEQ ID NO: 1 and SEQ ID NO: 2, SEQ ID NO: 3 and SEQ ID NO: 4, SEQ ID NO: 5 and SEQ ID NO: 6, SEQ ID NO: 7 and SEQ ID NO: 8, SEQ ID NO: 9 and SEQ ID NO:10, SEQ ID NO: 11 and SEQ ID NO: 12, SEQ ID NO: 13 and SEQ ID NO: 14, SEQ ID NO: 15 and SEQ ID NO: 16, SEQ ID NO: 17 and SEQ ID NO: 18, SEQ ID NO: 19 and SEQ ID NO: 20, SEQ ID NO: 21 and SEQ ID NO: 22, SEQ ID NO: 23 and SEQ ID NO: 24, SEQ ID NO: 25 and SEQ ID NO: 26, SEQ ID NO: 27 and SEQ ID NO: 28, SEQ ID NO: 29 and SEQ ID NO: 30, SEQ ID NO: 31 and SEQ ID NO: 32, SEQ ID NO: 33 and SEQ ID NO: 34, SEQ ID NO: 35 and SEQ ID NO: 36, SEQ ID NO: 37 and SEQ ID NO: 38, SEQ ID NO: 39 and SEQ ID NO: 40, SEQ ID NO: 41 and SEQ ID NO: 42, SEQ ID NO: 43 and SEQ ID NO: 44, SEQ ID NO: 45 and SEQ ID NO: 46, SEQ ID NO: 47 and SEQ ID NO: 48, SEQ ID NO: 49 and SEQ ID NO: 50, SEQ ID NO: 51 and SEQ ID NO: 52, SEQ ID NO: 53 and SEQ ID NO: 54, SEQ ID NO: 55 and SEQ ID NO: 56, SEQ ID NO: 57 and SEQ ID NO: 58, SEQ ID NO: 59 and SEQ ID NO: 60, SEQ ID NO: 61 and SEQ ID NO: 62, SEQ ID NO: 63 and SEQ ID NO: 64, SEQ ID NO: 65 and SEQ ID NO: 66, SEQ ID NO: 67 and SEQ ID NO: 68, SEQ ID NO: 69 and SEQ ID NO: 70, SEQ ID NO: 71 and SEQ ID NO: 72, SEQ ID NO: 73 and SEQ ID NO: 74, SEQ ID NO: 75 and SEQ ID NO: 76, SEQ ID NO: 79 and SEQ ID NO: 80, and SEQ ID NO: 81 and SEQ ID NO: 82 (i.e., T4.1).

3. The pharmaceutical composition of claim 1, wherein the isolated antibody or an immunologically active fragment that specifically binds human CD47 comprises a variable heavy (VH) chain sequence that is at least 95% identical to an amino acid sequence selected from the group consisting of SEQ ID NO: 3, SEQ ID NO: 31, SEQ ID NO. 79, and SEQ ID NO: 81; and a variable light (VL) chain sequence that is at least 95% identical to an amino acid sequence selected from the group consisting of SEQ ID NO: 4, SEQ ID NO: 32, SEQ ID NO: 80 and SEQ ID NO: 82.

4. The pharmaceutical composition of any one of claims 1-3, wherein the isolated antibody or an immunologically active fragment that specifically binds human CD47 comprises a VH/VL pair, wherein the VH/VL pair comprises VH and VL chain sequences that are respectively at least 95% identical to amino acid sequences selected from the group consisting of SEQ ID NO: 3 and SEQ ID NO: 4, SEQ ID NO: 31 and SEQ ID NO: 32, SEQ ID NO: 79 and SEQ ID NO: 80, and SEQ ID NO: 81 and SEQ ID NO: 82.

5. The pharmaceutical composition of any one of claims 1-4, wherein the isolated antibody or an immunologically active fragment thereof that specifically binds human CD47 comprises a heavy chain of SEQ ID NO: 81 and a light chain of SEQ ID NO: 82.

6. A pharmaceutical composition comprising an isolated antibody or an immunologically active fragment thereof that specifically binds human CD47, a therapeutic agent, and a pharmaceutically acceptable carrier, wherein the isolated antibody or an immunologically active fragment thereof that specifically binds human CD47 comprises
a heavy chain variable region (VH) having CDR1 of the amino acid sequence of NAWMN (SEQ ID NO: 85) or RAWMN (SEQ ID NO: 86), CDR2 having the amino acid sequence of RIKRKTDGETTDYAAPVKG (SEQ ID NO: 87), and CDR3 having the amino acid sequence of SNRAFDI (SEQ ID NO: 88), and
a light chain variable region (VL) having CDR1 of the amino acid sequence of KSSQSVLYSSNNRNYLA (SEQ ID NO: 89) or KSSQSVLYAGNNRNYLA (SEQ ID NO: 90), CDR2 of the amino acid sequence of QASTRAS (SEQ ID NO: 91), and CDR3 of the amino acid sequence of QQYYTPPLA (SEQ ID NO: 92).

7. The pharmaceutical composition of claim 6, wherein the isolated antibody or an immunologically active fragment that specifically binds human CD47 comprises a VH/VL pair, wherein the VH/VL pair comprises VH and VL chain sequences that are respectively at least 95% identical to amino acid sequences selected from the group consisting of SEQ ID NO: 3 and SEQ ID NO: 4, SEQ ID NO: 31 and SEQ ID NO: 32, SEQ ID NO: 79 and SEQ ID NO: 80, and SEQ ID NO: 81 and SEQ ID NO: 82.

8. The pharmaceutical composition of claim 6 or 7, where the isolated antibody or an immunologically active fragment thereof that specifically binds human CD47 comprises a heavy chain of SEQ ID NO: 81 and a light chain of SEQ ID NO: 82.

9. The pharmaceutical composition of any one of claims 1-8, wherein the isolated antibody or an immunologically active fragment thereof that specifically binds human CD47 is chimeric or humanized.

10. The pharmaceutical composition of any one of claims 1-9, wherein the isolated antibody or an immunologically active fragment thereof that specifically binds human CD47 is a monoclonal antibody, a bispecific antibody or a fusion antibody.

11. The pharmaceutical composition of claim 10, wherein the isolated bispecific antibody comprises a first arm and a second arm, wherein the first arm comprises the antibody or an immunologically active fragment thereof of any one of claims 1-4 which specifically binds human CD47, and the second arm comprises a second monoclonal antibody that does not bind human CD47.

12. The pharmaceutical composition of claim 11, wherein the second arm of the isolated bispecific antibody binds to a cancer cell.

13. The pharmaceutical composition of claim 10, wherein the fusion antibody is the isolated antibody or an immunologically active fragment thereof of claim 1 conjugated with an additional protein, a small-molecule agent or a marker.

14. The pharmaceutical composition of claim 13, wherein the additional protein in the fusion protein is an antibody or a cytokine; wherein the small molecule agent in the fusion protein is an anti-cancer or anti-inflammation agent; wherein the marker is a biomarker or fluorescent marker.

15. The pharmaceutical composition of any one of claims 1-14, wherein the isolated antibody or an immunologically active fragment thereof prevents human CD47 from interacting with signal-regulatory-protein α (SIRPα).

16. The pharmaceutical composition of any one of claims 1-12, wherein the isolated antibody or an immunologically active fragment thereof promotes macrophage-mediated phagocytosis of a CD47-expressing cell.

17. The pharmaceutical composition of any one of claims 1-16, wherein the isolated antibody or an immunologically active fragment thereof does not cause a significant level of hemagglutination or depletion of red blood cells.

18. The pharmaceutical composition of any one of claims 1-17, wherein the therapeutic agent synergizes the effect of the isolated antibody or an immunologically active fragment thereof that specifically binds human CD47 to promote phagocytosis.

19. The pharmaceutical composition of any one of claims 1-18, wherein the pharmaceutical composition is a synergistic combination, and the therapeutic agent enhances the therapeutic effect of the isolated antibody or immunologically active fragment thereof on phagocytic elimination of cancer cell.

20. The pharmaceutical composition of any one of claims 1-19, wherein the therapeutic agent increases expression level of Calreticulin.

21. The pharmaceutical composition of any one of claims 1-20, wherein the therapeutic agent inhibits expression level of CD47.

22. The pharmaceutical composition of any one of claims 1-21, wherein the therapeutic agent does not cause substantial toxicity to macrophages.

23. The pharmaceutical composition of any one of claims 1-22, wherein the therapeutic agent is a chemotherapeutic agent.

24. The pharmaceutical composition of claim 23, wherein the chemotherapeutic agent comprises Azacitidine, Venetoclax, or Copanilisib.

25. The pharmaceutical composition of claim 23 or 24, wherein the chemotherapeutic agent comprises Azacitidine or Venetoclax.

26. The pharmaceutical composition of any one of claims 23-25, wherein the chemotherapeutic agent comprises Azacitidine.

27. The pharmaceutical composition of any of claims 1-22, wherein the therapeutic agent is an antibody or an immunologically active fragment thereof that selectively binds CD20 and promotes phagocytic elimination of cancer cell.

28. The pharmaceutical composition of claim 27, wherein the antibody or an immunologically active fragment thereof that selectively binds CD20 is Rituximab or a biosimilar thereof.

29. The pharmaceutical composition of any one of claims 1-22, wherein the therapeutic agent comprises a chemotherapeutic agent and an antibody or an immunologically active fragment thereof that selectively binds CD20.

30. The pharmaceutical composition of any one of claims 27-29, wherein the isolated antibody or an immunologically active fragment that binds CD47 synergizes with the antibody selectively binding CD20 and promotes phagocytic elimination of cancer cell.

31. The pharmaceutical composition of claim 30, wherein the therapeutic agent comprises Azacitidine and Rituximab, or Venetoclax and Rituximab.

32. A method for treating a disease in a human subject in need thereof, comprising administering to the subject a pharmaceutical composition of any one of claims 1-31 in a therapeutically effective amount, wherein the disease is cancer, a fibrotic disease, a disease related to inhibition of phagocytosis, or a disease related to platelet aggregation.

33. The method of claim 32, wherein the cancer is selected from the group consisting of ovarian cancer, colon cancer, breast cancer, lung cancer, head and neck cancer, bladder cancer, colorectal cancer, pancreatic cancer, non-Hodgkin's lymphoma, acute lymphocytic leukemia, chronic lymphocytic leukemia, acute myeloid leukemia, chronic myelogenous leukemia, hairy cell leukemia (HCL), T-cell prolymphocytic leukemia (T-PLL), large granular lymphocytic leukemia, adult T-cell leukemia, multiple myeloma, melanoma, leiomyoma, leiomyosarcoma, glioma, glioblastoma, myelomas, monocytic leukemias, B-cell derived leukemias, T-cell derived leukemias, B-cell derived lymphomas, T-cell derived lymphomas, endometrial cancer, kidney cancer, melanoma, prostate cancer, thyroid cancer, cervical cancer, gastric cancer, liver cancer, and solid tumors; the fibrotic disease is selected from the group consisting of: myocardial infarction, angina, osteoarthritis, pulmonary fibrosis, asthma, cystic fibrosis, bronchitis, and asthma; the disease related to inhibition of phagocytosis is a cardiovascular disease; the disease related to platelet aggregation is Glanzmann Thrombasthenia, prolonged bleeding time, immune thrombocytopenia (ITP), von Willebrand disease (vWD).

34. The method of claim 32 or 33, wherein the cardiovascular disease is selected from the group consisting of atherosclerosis, stroke, hypertensive heart disease, rheumatic heart disease, cardiomyopathy, heart arrhythmia, congenital heart disease, valvular heart disease, carditis, aortic aneurysms, peripheral artery disease, and venous thrombosis.

35. Use of a pharmaceutical composition of any one of claims 1-31 for the manufacture of a medicament for treatment of a disease, wherein the disease is cancer, a fibrotic disease, a disease related to inhibition of phagocytosis, or a disease related to platelet aggregation.


SEQUENCE LISTING (92 sequences)

SEQ ID NO: 1 - protein, 119 aa, Homo sapiens
QVQLQQSGGGLVQPGGSLRLSCTASGFTFSSYAMSWVRQAPGKGLEWVSAISGSGGSTYYADSVKGRFTISRDNSKNTLYLQMNSLRVEDTAVYYCARYSIGRHTFDHWGQGTLVTVSA

SEQ ID NO: 2 - protein, 111 aa, Homo sapiens
NFMLTQPHSVSESPGKTVTISCTRSSGGIASNFVQWYQQRPGSVPTTVIYRDNQRPSGVPDRFSGSVDSSSNSASLTISGLKTDDEADYYCQSYDDHNHWVFGGGTKLTVL

SEQ ID NO: 3 - protein, 118 aa, Homo sapiens
KVQLVESGGGLVKPGGSLRLSCAASGFTFSNAWMNWVRQAPGKGLEWVGRIKRKTDGETTDYAAPVKGRFSISRDDSKNTLYLQMNSLKTEDTAVYYCAGSNRAFDIWGQGTMVTVSA

SEQ ID NO: 4 - protein, 113 aa, Homo sapiens
DIVMTQSPDSLAVSLGERATINCKSSQSVLYSSNNRNYLAWYQQKPGQPPKLLINQASTRASGVPDRFSGSGSGTEFTLIISSLHAEDVAIYYCQQYYTPPLAFGGGTKLEIK

SEQ ID NO: 5 - protein, 118 aa, Homo sapiens
QVQLQQSGGGLVQPGGSLRLSCAASGFTFSGYAMTWVRQAPGKGLEWVSAITSTGGRTYYADSVKGRFTTSRDNSRNTLYLQMNSLRAEDTAVYYCARESNFRAFDIWGQGTMVTVSA

SEQ ID NO: 6 - protein, 112 aa, Homo sapiens
EIVLTQSPLSLPVTPGEPASISCRSSQSLLHSNGYNYLDWYLQKPGQSPQLLIYLNSNRASGVPDRFSGSGSGTDFTLQISRVEAEDVGVYYCMQALQIPPTFGGGTKVDIK

SEQ ID NO: 7 - protein, 118 aa, Homo sapiens
EVQLVESGGGLVQPGGSLRLSCAASGFTFIDAWMTWVRQAPGKGLEWVSVIYSGGSTYYADSVKGRFTISRDNSKNTLYLQMNSLRAEDTAVYYCARGARGHPGQDYWGQGTLVTVSA

SEQ ID NO: 8 - protein, 112 aa, Homo sapiens
NFMLTQPHSVSESPGKTVTISCTRSSGTIASNFVQWYQQRPGSSPTPVIFENDRRPSGVPDRFSGSIDSSSNSASLTISSLNTEDKADYYCQSYDSSTHGWVFGGGTQLTVL

SEQ ID NO: 9 - protein, 117 aa, Homo sapiens
QVNLRESGGGLVQPGGSLRLSCAASGFTFSDYYMSWIRQAPGGGLEWVSYTSRFGSDTNYADSVKGRFTISRDNVQNSLYLQMNSLRAEDTAVYYCVRDVHNRDAYWGQGTLVTVSA

SEQ ID NO: 10 - protein, 110 aa, Homo sapiens
SYVLTQPPSASGTPGQRVTISCSGSSSNIGGNSVSWYQQLPGTAPKLLIYRNHQRPSGVPDRFSGSKSGTSASLAISGLRSDDEADYYCATWDFSLSGFVFGTGTKVTVL

SEQ ID NO: 11 - protein, 108 aa, Homo sapiens
QVQLVQSGGGLVQPGGSLRLSCAASGFTFSSYAMSWVRQAPGKGLEWVSAISGSGGSTYYADSVKGRFTISRDNSKNTYYCANTDYYDSSSHTPADYWGQGTLVTVSA

SEQ ID NO: 12 - protein, 108 aa, Homo sapiens
ETTLTQSPSSLSASVGDRVTITCRASQDIRNDLDWFQQKPGEAPKRLISAASNLQSGVPSRFSGGGSGSEFTLTIHSLESEDFATYYCQQSYITPPWTFGQGTKLEIK

SEQ ID NO: 13 - protein, 122 aa, Homo sapiens
QVQLQESGGGLVQPGGSLRLSCAASGFTFSSYGMSWVRQAPGKGLEWVSTISGSGSSTNYADSVKGRFTISRDNSKNTLFLQMNSLRAEDTAVYFCAKGRYYYDSLDAFDIWGQGTMVTVSA

SEQ ID NO: 14 - protein, 108 aa, Homo sapiens
EIVLTQSPGTLSLSPGERATLSCRASQEIRTAYLAWYQQKPGQAPRLLIYYASSRATGIPDRFSGSRSGTDFTLTISRLEPEDFAVYSCQQYDTSPPTFGQGTRLEIK

SEQ ID NO: 15 - protein, 122 aa, Homo sapiens
QVQLVESGGGLVQPGGSLRLSCAASGFTFSSYAMSWVRQAPGKGLEWVSAISGTGGSTYYADSVKGRFTISRDNSKNTLYLQMNSLRAEDTAAYYCAKDKWSSWPTYYFDYWGQGTLVTVSA

SEQ ID NO: 16 - protein, 110 aa, Homo sapiens
NFMLTQPHSVSESPGKTVTISCTRSSGSIASNYVQWYQQRPGSSPTTVIYEDNQRPSGVPDRFSGSIDSSSNSASLTISGLKTEDEADYYCQSYDSSNVIFGGGTKVTVL

SEQ ID NO: 17 - protein, 122 aa, Homo sapiens
QVQLQVSGGGLVQPGGSLRLSCAASGFTFSSYSMAWVRQAPGKGLEWVAAVSNSGVETYYADSVKGRFTISRDNSKNTLYLQMNSLRAEDTAVYYCAKRTRQLLTPREFDYWGQGTLVTVLA

SEQ ID NO: 18 - protein, 107 aa, Homo sapiens
ETTLTQSPSSVSASVGDRVTLTCRASQDITRWLAWYQQKPGKAPKLLIYDASSLQSGVPSRFSGSGSGTDFTLTISSLQPEDFATYYCQQGSSVPFTFGGGTKVEIK

SEQ ID NO: 19 - protein, 117 aa, Homo sapiens
QVNLRESGGGLIQPGGSLRLSCAASGFTFTNYAMSWVRQAPGKGLEWVSSVSSAGGSTYYADSVKGRFTISRDNAKNSLYLQMNSLRAEDTAVYYCARRVNRAFDLWGRGTLVTVSA

SEQ ID NO: 20 - protein, 110 aa, Homo sapiens
DVVMTQSPGTLSLSPGERATLSCRASQSVSSSYLAWYQQKPGQAPRLLIYGASSRATGIPDRFSGSGSGTDFTLTISRLEPEDFAVYYCQQYGSSPPMYTFGQGTKLEIK

SEQ ID NO: 21 - protein, 121 aa, Homo sapiens
QVQLQESGGGLVQPGGSLRLSCAASGFTFSNAWMSWVRQAPGKGLVWVGRIKSKTDGGTTDYAAPVKGRFTISRDDSKNTLYLQMNSLKTEDTAVYYCTTDKSYGYTFDYWGQGTLVTVSA

SEQ ID NO: 22 - protein, 110 aa, Homo sapiens
SYVLTQPPSASGTPGQRVTISCSGSGSNIGSNSVHWYQQLPGTAPKLLIYTNNQRPSGVPDRFSGSKSGISASLAISGLQSEDEAVYYCATWDDRLSGPVFAAGTKLTVL

SEQ ID NO: 23 - protein, 119 aa, Homo sapiens
QVNLRESGGGLVKPGGSLRLSCAASGFTFSSYWMHWVRQAPGKGLEWVSAISGSGAGTYYPDSVKGRFTISRDNSKNTLYLQMNSLRAEDTAVYYCARDRSLSFGFDIWGQGTLVSVSG

SEQ ID NO: 24 - protein, 110 aa, Homo sapiens
NFMLTQPHSVSGSPGKTVTISCTRSSGSIGSTYVQWYQQRPGSPPTTVIYKDDQRPSGVPDRFSGSIDGSSNSASLTISGLETEDEADYYCQSSDTSNLVFGGGTKVTVL

SEQ ID NO: 25 - protein, 121 aa, Homo sapiens
QVQLQQSGGGLVQPGGSLRLSCAAPGFTFSRYWMSWVRQAPGKGLEWVANIKGDGSQTYYADSVKGRFTISRDNAMKTVYLQMNSLRAEDTAIYYCAKGAAYHINSWLDPWGQGTLVTVSA

SEQ ID NO: 26 - protein, 108 aa, Homo sapiens
ETTLTQSPGTLSVSPGERVTLSCRASQSISGNYLAWYQQRPGQAPRLLIYGAFRRATGIPDRFSGSGSGTDFTLTITRLEPEDFATYYCQHYNNFPHTFGAGTKVDIK

SEQ ID NO: 27 - protein, 118 aa, Homo sapiens
KVQLVESGGGLVKPGGSLRLSCAASGITFKHAWMNWVRQAPGKGLEWVGRIKRKTDGETTDYAAPVKGRFSISRDDSKNTLYLQMNSLKTEDTAVYYCAGSNRAFDIWGQGTMVTVSA

SEQ ID NO: 28 - protein, 113 aa, Homo sapiens
DIVMTQSPDSLAVSLGERATINCKSSQSVLYQVNNRNYLAWYQQKPGQPPKLLINQASTRASGVPDRFSGSGSGTEFTLIISSLQAEDVAIYYCQQYYTPPLAFGGGTKLEIK

SEQ ID NO: 29 - protein, 118 aa, Homo sapiens
KVQLVESGGGLVKPGGSLRLSCAASGFTFNNAWMNWVRQAPGKGLEWVGRIKRKTDGETTDYAAPVKGRFSISRDDSKNTLYLQMNSLKTEDTAVYYCAGSNRAFDIWGQGTMVTVSA

SEQ ID NO: 30 - protein, 113 aa, Homo sapiens
DIVMTQSPDSLAVSLGERATINCKSSQTVLYPLNNRNYLAWYQQKPGQPPKLLINQASTRASGVPDRFSGSGSGTEFTLIISSLQAEDVAIYYCQQYYTPPLAFGGGTKLEIK

SEQ ID NO: 31 - protein, 118 aa, Homo sapiens
KVQLVESGGGLVKPGGSLRLSCAASGLTFERAWMNWVRQAPGKGLEWVGRIKRKTDGETTDYAAPVKGRFSISRDDSKNTLYLQMNSLKTEDTAVYYCAGSNRAFDIWGQGTMVTVSA

SEQ ID NO: 32 - protein, 113 aa, Homo sapiens
DIVMTQSPDSLAVSLGERATINCKSSQSVLYAGNNRNYLAWYQQKPGQPPKLLINQASTRASGVPDRFSGSGSGTEFTLIISSLQAEDVAIYYCQQYYTPPLAFGGGTKLEIK

SEQ ID NO: 33 - protein, 118 aa, Homo sapiens
KVQLVESGGGLVKPGGSLRLSCAASGFTFPNAWMNWVRQAPGKGLEWVGRIKRKTDGETTDYAAPVKGRFSISRDDSKNTLYLQMNSLKTEDTAVYYCAGSNRAFDIWGQGTMVTVSA

SEQ ID NO: 34 - protein, 113 aa, Homo sapiens
DIVMTQSPDSLAVSLGERATINCKSSQSVLYPGNNRNYLAWYQQKPGQPPKLLINQASTRASGVPDRFSGSGSGTEFTLIISSLQAEDVAIYYCQQYYTPPLAFGGGTKLEIK

SEQ ID NO: 35 - protein, 118 aa, Homo sapiens
KVQLVESGGGLVKPGGSLRLSCAASGLTFGNAWMNWVRQAPGKGLEWVGRIKRKTDGETTDYAAPVKGRFSISRDDSKNTLYLQMNSLKTEDTAVYYCAGSNRAFDIWGQGTMVTVSA

SEQ ID NO: 36 - protein, 113 aa, Homo sapiens; identical to SEQ ID NO: 34

SEQ ID NO: 37 - protein, 118 aa, Homo sapiens
KVQLVESGGGLVKPGGSLRLSCAASGFTFSNAWMNWVRQAPGKGLEWVGRIKRKTDGETTDYAAPVKGRFSISRDDSKNTLYLQMNSLKTEDTAVYYCAGGNHSSDIWGQGTMVTVSA

SEQ ID NO: 38 - protein, 113 aa, Homo sapiens
DIVMTQSPDSLAVSLGERATINCKSSQSVLYSSNNRNYLAWYQQKPGQPPKLLINQASTRASGVPDRFSGSGSGTEFTLIISSLQAEDVAIYYCQQYYTPPLAFGGGTKLEIK

SEQ ID NO: 39 - protein, 118 aa, Homo sapiens
KVQLVESGGGLVKPGGSLRLSCAASGFTFSNAWMNWVRQAPGKGLEWVGRIKRKTDGETTDYAAPVKGRFSISRDDSKNTLYLQMNSLKTEDTAVYYCAGGAHSSDIWGQGTMVTVSA

SEQ ID NO: 40 - protein, 113 aa, Homo sapiens; identical to SEQ ID NO: 38

SEQ ID NO: 41 - protein, 118 aa, Homo sapiens
KVQLVESGGGLVKPGGSLRLSCAASGFTFSNAWMNWVRQAPGKGLEWVGRIKRKTDGETTDYAAPVKGRFSISRDDSKNTLYLQMNSLKTEDTAVYYCAGGQHSSDIWGQGTMVTVSA

SEQ ID NO: 42 - protein, 113 aa, Homo sapiens; identical to SEQ ID NO: 38

SEQ ID NO: 43 - protein, 118 aa, Homo sapiens
KVQLVESGGGLVKPGGSLRLSCAASGFTFSNAWMNWVRQAPGKGLEWVGRIKRKTDGETTDYAAPVKGRFSISRDDSKNTLYLQMNSLKTEDTAVYYCAGSAYAFDAWGQGTMVTVSA

SEQ ID NO: 44 - protein, 113 aa, Homo sapiens; identical to SEQ ID NO: 38

SEQ ID NO: 45 - protein, 118 aa, Homo sapiens
KVQLVESGGGLVKPGGSLRLSCAASGFTFSNAWMNWVRQAPGKGLEWVGRIKRKTDGETTDYAAPVKGRFSISRDDSKNTLYLQMNSLKTEDTAVYYCAGSAYAFDSWGQGTMVTVSA

SEQ ID NO: 46 - protein, 113 aa, Homo sapiens; identical to SEQ ID NO: 38

SEQ ID NO: 47 - protein, 118 aa, Homo sapiens
KVQLVESGGGLVKPGGSLRLSCAASGFTFSNAWMNWVRQAPGKGLEWVGRIKRKTDGETTDYAAPVKGRFSISRDDSKNTLYLQMNSLKTEDTAVYYCAGSDRASDKWGQGTMVTVSA

SEQ ID NO: 48 - protein, 113 aa, Homo sapiens; identical to SEQ ID NO: 38

SEQ ID NO: 49 - protein, 118 aa, Homo sapiens
KVQLVESGGGLVKPGGSLRLSCAASGFTFSNAWMNWVRQAPGKGLEWVGRIKRKTDGETTDYAAPVKGRFSISRDDSKNTLYLQMNSLKTEDTAVYYCAGSAYAFDTWGQGTMVTVSA

SEQ ID NO: 50 - protein, 113 aa, Homo sapiens; identical to SEQ ID NO: 38

SEQ ID NO: 51 - protein, 118 aa, Homo sapiens
KVQLVESGGGLVKPGGSLRLSCAASGFTFSNAWMNWVRQAPGKGLEWVGRIKRKTDGETTDYAAPVKGRFSISRDDSKNTLYLQMNSLKTEDTAVYYCAGGNHSQDIWGQGTMVTVSA

SEQ ID NO: 52 - protein, 113 aa, Homo sapiens; identical to SEQ ID NO: 38

SEQ ID NO: 53 - protein, 118 aa, Homo sapiens
KVQLVESGGGLVKPGGSLRLSCAASGFTFSNAWMNWVRQAPGKGLEWVGRIKRKTDGETTDYAAPVKGRFSISRDDSKNTLYLQMNSLKTEDTAVYYCAGGQHSQDIWGQGTMVTVSA

SEQ ID NO: 54 - protein, 113 aa, Homo sapiens; identical to SEQ ID NO: 38

SEQ ID NO: 55 - protein, 118 aa, Homo sapiens
KVQLVESGGGLVKPGGSLRLSCAASGFTFSNAWMNWVRQAPGKGLEWVGRIKRKTDGETTDYAAPVKGRFSISRDDSKNTLYLQMNSLKTEDTAVYYCAGGAHSQDIWGQGTMVTVSA

SEQ ID NO: 56 - protein, 113 aa, Homo sapiens; identical to SEQ ID NO: 38

SEQ ID NO: 57 - protein, 118 aa, Homo sapiens; identical to SEQ ID NO: 3

SEQ ID NO: 58 - protein, 113 aa, Homo sapiens
DIVMTQSPDSLAVSLGERATINCKSSQSVLYSSNNRNYLAWYQQKPGQPPKLLINQASTRASGVPDRFSGSGSGTEFTLIISSLQAEDVAIYYCQQYLTPPLAFGGGTKLEIK

SEQ ID NO: 59 - protein, 118 aa, Homo sapiens; identical to SEQ ID NO: 3

SEQ ID NO: 60 - protein, 113 aa, Homo sapiens
DIVMTQSPDSLAVSLGERATINCKSSQSVLYSSNNRNYLAWYQQKPGQPPKLLINQASTRASGVPDRFSGSGSGTEFTLIISSLQAEDVAIYYCQQYLRPPLNFGGGTKLEIK

SEQ ID NO: 61 - protein, 118 aa, Homo sapiens; identical to SEQ ID NO: 3

SEQ ID NO: 62 - protein, 113 aa, Homo sapiens
DIVMTQSPDSLAVSLGERATINCKSSQSVLYSSNNRNYLAWYQQKPGQPPKLLINQASTRASGVPDRFSGSGSGTEFTLIISSLQAEDVAIYYCQQYLTPPLNFGGGTKLEIK

SEQ ID NO: 63 - protein, 118 aa, Homo sapiens; identical to SEQ ID NO: 3

SEQ ID NO: 64 - protein, 113 aa, Homo sapiens
DIVMTQSPDSLAVSLGERATINCKSSQSVLYSSNNRNYLAWYQQKPGQPPKLLINQASTRASGVPDRFSGSGSGTEFTLIISSLQAEDVAIYYCQNYLTPPLSFGGGTKLEIK

SEQ ID NO: 65 - protein, 118 aa, Homo sapiens; identical to SEQ ID NO: 3

SEQ ID NO: 66 - protein, 113 aa, Homo sapiens
DIVMTQSPDSLAVSLGERATINCKSSQSVLYSSNNRNYLAWYQQKPGQPPKLLINQASTRASGVPDRFSGSGSGTEFTLIISSLQAEDVAIYYCQQYLKAPLAFGGGTKLEIK

SEQ ID NO: 67 - protein, 118 aa, Homo sapiens; identical to SEQ ID NO: 3

SEQ ID NO: 68 - protein, 113 aa, Homo sapiens
DIVMTQSPDSLAVSLGERATINCKSSQSVLYSSNNRNYLAWYQQKPGQPPKLLINQASTRASGVPDRFSGSGSGTEFTLIISSLQAEDVAIYYCQQYLNAPLHFGGGTKLEIK

SEQ ID NO: 69 - protein, 118 aa, Homo sapiens; identical to SEQ ID NO: 3

SEQ ID NO: 70 - protein, 113 aa, Homo sapiens
DIVMTQSPDSLAVSLGERATINCKSSQSVLYSSNNRNYLAWYQQKPGQPPKLLINQASTRASGVPDRFSGSGSGTEFTLIISSLQAEDVAIYYCQQYLEAPLVFGGGTKLEIK

SEQ ID NO: 71 - protein, 118 aa, Homo sapiens; identical to SEQ ID NO: 3

SEQ ID NO: 72 - protein, 113 aa, Homo sapiens
DIVMTQSPDSLAVSLGERATINCKSSQSVLYSSNNRNYLAWYQQKPGQPPKLLINQASTRASGVPDRFSGSGSGTEFTLIISSLQAEDVAIYYCQQYLKAPLHFGGGTKLEIK

SEQ ID NO: 73 - protein, 118 aa, Homo sapiens; identical to SEQ ID NO: 3

SEQ ID NO: 74 - protein, 113 aa, Homo sapiens
DIVMTQSPDSLAVSLGERATINCKSSQSVLYSSNNRNYLAWYQQKPGQPPKLLINQASTRASGVPDRFSGSGSGTEFTLIISSLQAEDVAIYYCQRLIAPPFTFGGGTKLEIK

SEQ ID NO: 75 - protein, 118 aa, Homo sapiens; identical to SEQ ID NO: 3

SEQ ID NO: 76 - protein, 113 aa, Homo sapiens
DIVMTQSPDSLAVSLGERATINCKSSQSVLYSSNNRNYLAWYQQKPGQPPKLLINQASTRASGVPDRFSGSGSGTEFTLIISSLQAEDVAIYYCQNYLTPPLAFGGGTKLEIK

SEQ ID NO: 77 - protein, 120 aa, Homo sapiens
QVQLVQSGAEVKKPGSSVKVSCKASGYTFSSYYMHWVRQAPGQGLEWMGEINPNNARINFNEKFKTRVTLTVDKSTSTAYMELSSLRSEDTAVYYCTRGYYRYGAWFGYWGQGTLVTVSS

SEQ ID NO: 78 - protein, 107 aa, Homo sapiens
DIQMTQSPSSLSASVGDRVTITCRASQDISDYLNWYQQKPGKAPKLLIYYISRLHSGVPSRFSGSGSGTDYTLTISSLQPEDFATYYCQQGHTLPWTFGGGTKVEIK

SEQ ID NO: 79 - protein, 118 aa, Homo sapiens
EVQLVESGGGLVKPGGSLRLSCAASGLTFERAWMNWVRQAPGKGLEWVGRIKRKTDGETTDYAAPVKGRFSISRDDSKNTLYLQMNSLKTEDTAVYYCAGSNRAFDIWGQGTMVTVSS

SEQ ID NO: 80 - protein, 113 aa, Homo sapiens; identical to SEQ ID NO: 32

SEQ ID NO: 81 - protein, 445 aa, Homo sapiens
EVQLVESGGGLVKPGGSLRLSCAASGLTFERAWMNWVRQAPGKGLEWVGRIKRKTDGETTDYAAPVKGRFSISRDDSKNTLYLQMNSLKTEDTAVYYCAGSNRAFDIWGQGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 82 - protein, 220 aa, Homo sapiens
DIVMTQSPDSLAVSLGERATINCKSSQSVLYAGNNRNYLAWYQQKPGQPPKLLINQASTRASGVPDRFSGSGSGTEFTLIISSLQAEDVAIYYCQQYYTPPLAFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 83 - protein, 451 aa, Homo sapiens
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 84 - protein, 213 aa, Homo sapiens
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 85 - protein, 5 aa, Homo sapiens
NAWMN

SEQ ID NO: 86 - protein, 5 aa, Homo sapiens
RAWMN

SEQ ID NO: 87 - protein, 19 aa, Homo sapiens
RIKRKTDGETTDYAAPVKG

SEQ ID NO: 88 - protein, 7 aa, Homo sapiens
SNRAFDI

SEQ ID NO: 89 - protein, 17 aa, Homo sapiens
KSSQSVLYSSNNRNYLA

SEQ ID NO: 90 - protein, 17 aa, Homo sapiens
KSSQSVLYAGNNRNYLA

SEQ ID NO: 91 - protein, 7 aa, Homo sapiens
QASTRAS

SEQ ID NO: 92 - protein, 9 aa, Homo sapiens
QQYYTPPLA